(19) United States
(12) Patent Application Publication
(10) Pub. No.: US 2022/0215625 A1
(43) Pub. Date: Jul. 7, 2022

(54) IMAGE-BASED METHODS FOR ESTIMATING A PATIENT-SPECIFIC REFERENCE BONE MODEL FOR A PATIENT WITH A CRANIOMAXILLOFACIAL DEFECT AND RELATED SYSTEMS

(71) Applicants: THE METHODIST HOSPITAL SYSTEM, Houston, TX (US); THE UNIVERSITY OF NORTH CAROLINA AT CHAPEL HILL, Chapel Hill, NC (US)

(72) Inventors: James Jiong XIA, Houston, TX (US); Jaime GATENO, Bellaire, TX (US); Dinggang SHEN, Chapel Hill, NC (US)

(21) Appl. No.: 17/600,909

(22) PCT Filed: Apr. 2, 2020
(86) PCT No.: PCT/US2020/026408

§ 371 Date: Oct. 1, 2021

Related U.S. Application Data
(60) Provisional application No. 62/828,048, filed on Apr. 2, 2019.

(51) Int. Cl. G06T 17/20; A61B 34/10; A61B 6/03; A61B 6/00; G06T 19/20; G06T 7/33

(52) U.S. Cl. CPC G06T 17/20; A61B 34/10; A61B 6/032; A61B 6/501; G06T 19/20; G06T 7/344; G06T 2207/30008; G06T 2210/41; G06T 2219/2004; G06T 2207/30201; G06T 2207/20084; G06T 2219/2021; G06T 2207/10081; A61B 2034/105

(57) ABSTRACT

Systems and methods for estimating a patient-specific reference bone shape model for a patient with craniomaxillofacial (CMF) defects are described herein. An example method includes receiving a twodimensional (“2D”) pre-trauma image of a subject, and generating a three-dimensional (“3D”) facial surface model for the subject from the 2D pre-trauma image. The method also includes providing a correlation model between 3D facial and bone surfaces, and estimating a reference bone model for the subject using the 3D facial surface model and the correlation model.

### CROSS-REFERENCE TO RELATED APPLICATIONS

This application claims the benefit of U.S. provisional patent application No. 62/828,048, filed on Apr. 2, 2019, and entitled “Multimodal Imaging and Photograph Guided Treatment Planning for Patients with Craniomaxillofacial Defects,” the disclosure of which is expressly incorporated herein by reference in its entirety.

### STATEMENT REGARDING FEDERALLY FUNDED RESEARCH

This invention was made with government support under Grant No. DE022676 and DE027251 awarded by the National Institutes of Health. The government has certain rights in the invention.

### BACKGROUND

Craniomaxillofacial (CMF) defects can be caused by traffic accidents, combat injuries, tumor ablations, etc. CMF reconstructive surgery is specifically designed to reconstruct such defects, by restoring the defected facial bones to a normal shape or a desired pre-traumatic status. The success of the CMF reconstructive surgery largely depends on accurate surgical planning. See R. B. Bell, “Computer planning and intraoperative navigation in cranio-maxillofacial surgery,” Oral and Maxillofacial Surgery Clinics, vol. 22, no. 1, pp. 135-156, 2010.

A computer-aided surgical simulation (CASS) platform for the treatment planning of various CMF surgeries has been developed. The CASS platform is described, for example, in J. J. Xia, et al., “Three-dimensional computer-aided surgical simulation for maxillofacial surgery,” Atlas Oral Maxillofac. Surg. Clin. North Am., vol. 13, no. 1, pp. 25-39, 2005; J. Gateno et al. Clinical feasibility of computer-aided surgical simulation (CASS) in the treatment of complex cranio-maxillofacial deformities. J Oral Maxillofac Surg. 2007 April; 65(4):728-34; J. J. Xia, et al., “New clinical protocol to evaluate craniomaxillofacial deformity and plan surgical correction,” J. Oral Maxillofac. Surg., vol. 67, no. 10, pp. 2093-2106, 2009; J. J. Xia, et al., “A new paradigm for complex midface reconstruction: a reversed approach,” J. Oral Maxillofac. Surg., vol. 67, no. 3, pp. 693-703, 2009; J. J. Xia, et al., “Algorithm for planning a double-jaw orthognathic surgery using a computer-aided surgical simulation (CASS) protocol. Part 1: planning sequence,” Int. J. Oral Maxillofacial Surg., vol. 44, no. 12, pp. 1431-1440, 2015; and P. Yuan, et al., “Design, development and clinical validation of computer-aided surgical simulation system for streamlined orthognathic surgical planning,” Int. J. Comput. Assist. Radiol. Surg., vol. 12, no. 12, pp. 2129-2143, 2017. In CASS, a computed tomography (CT) or cone-beam computed tomography (CBCT) scan is acquired for the reconstruction of the three-dimensional (3D) models of the patient's midface, mandible and face. After that, multiple 3D cephalometric measurements are quantified based on a group of anatomical landmarks manually digitized on the reconstructed head and face models. These cephalometric measurements for the patient are then compared with the normal ones (quantified from a set of normal subjects) to quantify patient's deformities. See J. Gateno, et al., “New 3-dimensional cephalometric analysis for orthognathic surgery,” J. Oral Maxillofac. Surg., vol. 69, no. 3, pp. 606-622, 2011; J. J. Xia, et al., “Algorithm for planning a double-jaw orthognathic surgery using a computer-aided surgical simulation (CASS) protocol. Part 2: three-dimensional cephalometry,” Int. J. Oral Maxillofac. Surg., vol. 44, no. 12, pp. 1441-1450, 2015. Based on the quantified deformities and surgeon's clinical experience, the surgery is finally simulated by virtually cutting the 3D model into multiple pieces and moving each of them to the desired positions.

Although the CASS platform described above works well in terms of both accuracy and cost efficiency in treating regular jaw deformities (see J. J. Xia, et al., “Outcome study of computer-aided surgical simulation in the treatment of patients with craniomaxillofacial deformities,” J. Oral Maxillofac. Surg., vol. 69, no. 7, pp. 2014-2024, 2011; J. J. Xia, et al., “Accuracy of the computer-aided surgical simulation (CASS) system in the treatment of patients with complex craniomaxillofacial deformity: A pilot study,”. J. Oral Maxillofac. Surg., vol. 65, no. 2, pp. 248-254, 2007; S. S. Hsu, et al., “Accuracy of a computer-aided surgical simulation protocol for orthognathic surgery: a prospective multicenter study,” J. Oral Maxillofac. Surg., vol. 71, no. 1, pp. 128-142, 2013; J. J. Xia, et al., “Cost-effectiveness analysis for computer-aided surgical simulation in complex cranio-maxillofacial surgery,” J. Oral Maxillofac. Surg., vol. 64, no. 12, pp. 1780-1784, 2006), it is challenging to produce ideal surgical outcomes for patients with complex CMF defects. One reason is that the CASS platform described above quantifies deformities subjectively, as it heavily depends on the surgeon's experience. Nonetheless, it is technically impossible to quantify and restore patient-specific, complex CMF defects (e.g., trauma) referring only to the simple mean numerical values of limited cephalometric evaluations on normal subjects.

The systems and methods described herein address the above limitations of the CASS platform, particularly when providing CMF surgical treatment planning for patients with complex CMF defects.

### SUMMARY

An example method for estimating a patient-specific reference bone shape model for a patient with CMF defects is described herein. The method includes receiving a two-dimensional (“2D”) pre-trauma image of a subject, and generating a three-dimensional (“3D”) facial surface model for the subject from the 2D pre-trauma image. The method also includes providing a correlation model between 3D facial and bone surfaces, and estimating a reference bone model for the subject using the 3D facial surface model and the correlation model.

Additionally, the 3D facial surface model is a 3D facial model of the subject's pre-trauma facial soft tissue.

Alternatively or additionally, the reference bone model is a 3D model of the subject's pre-trauma facial bone structure.

Optionally, the method further includes receiving a plurality of 2D pre-trauma images of the subject. The 3D facial surface model is generated from the plurality of 2D pre-trauma images. For example, a respective 3D facial surface model for the subject can be generated from each of the 2D pre-trauma images and the respective 3D facial surface models for the subject can be merged into a combined 3D facial surface model.

Alternatively or additionally, in some implementations, the step of generating a three-dimensional (“3D”) facial surface model for the subject from the 2D pre-trauma image includes analyzing the 2D pre-trauma image with a machine learning algorithm. For example, the method can include extracting, using a first machine learning algorithm, a plurality of landmarks from the 2D pre-trauma image, and generating, using a second machine learning algorithm, the 3D facial surface model from the extracted landmarks.

Alternatively or additionally, in some implementations, the step of generating a 3D facial surface model for the subject from the 2D pre-trauma image includes extracting, using a convolutional neural network (“CNN”), a plurality of landmarks from the 2D pre-trauma image, computing a sparse deformation field between the extracted landmarks and corresponding extracted landmarks for a reference 3D face model, converting the sparse deformation field into a dense deformation field, and applying the dense deformation field on the reference 3D face model to generate the 3D facial surface model.

Alternatively or additionally, the step of estimating a reference bone model for the subject using the 3D facial surface model and the correlation model includes registering the 3D facial surface model and a facial image template, mapping a plurality of sample points on the facial image template to the 3D facial surface model to obtain a plurality of corresponding sample points on the 3D facial surface model, and inputting a coordinate vector of the corresponding sample points into the correlation model to estimate the reference bone model for the subject. Optionally, the facial image template is selected from a plurality of facial surface models. Alternatively or additionally, the 3D facial surface model and the facial image template are registered using an iterative closest point (“ICP”) algorithm. Alternatively or additionally, the corresponding sample points on the 3D facial surface model are obtained using a coherent point drift (“CPD”) algorithm.

Optionally, the method further includes maintaining a database including a plurality of facial and bone surface models, where each of the facial and bone surface models is extracted from a 3D image of a reference subject, and establishing the correlation model based on the facial and bone surface models in the database. Optionally, the correlation model is established using a machine leaning algorithm such as sparse representation. Alternatively or additionally, the correlation model includes a face dictionary, which includes a matrix including a plurality of vectorized coordinates of a plurality of landmarks from each of the facial surface models. Alternatively or additionally, the correlation model includes a bone dictionary, which includes a matrix representing a plurality of vectorized coordinates of a plurality of landmarks on each of the bone surface models.

Optionally, the method further includes receiving a 3D post-trauma image of the subject, generating a 3D post-trauma bone model from the 3D post-trauma image, and refining the reference bone model by deforming the reference bone model onto the 3D post-trauma bone model. Additionally, the method optionally further includes constraining the reference bone model refinement using a statistical shape model (“SSM”). Alternatively or additionally, the reference bone model is refined using a machine learning algorithm such as an adaptive-focus deformable shape model (“AFDSM”). Optionally, the 3D post-trauma image is a computed-tomography (“CT”) image.

Alternatively or additionally, the 2D pre-trauma image is a digital photo.

Alternatively or additionally, the subject has CMF defects. Optionally, the CMF defects are bilateral.

Optionally, the method further includes using the reference bone model for reconstructive surgical planning.

An example system for estimating a patient-specific reference bone shape model for a patient with CMF defects is also described herein. The system includes a processing unit and a memory operably coupled to the processing unit. The memory has computer-executable instructions stored thereon that, when executed by the processing unit, cause the processing unit to receive a 2D pre-trauma image of a subject, generate a 3D facial surface model for the subject from the 2D pre-trauma image, provide a correlation model between 3D facial and bone surfaces, and estimate a reference bone model for the subject using the 3D facial surface model and the correlation model.

Another example system for estimating a patient-specific reference bone shape model for a patient with CMF defects is described herein. The system includes a first machine learning module configured to analyze 2D images, a second machine learning module configured to generate a model, a processing unit, and a memory operably coupled to the processing unit. The memory has computer-executable instructions stored thereon that, when executed by the processing unit, cause the processing unit to receive a 2D pre-trauma image of a subject, input the 2D pre-trauma image into the first machine learning module, where the first machine learning algorithm extracts a plurality of landmarks from the 2D pre-trauma image. The memory has further computer-executable instructions stored thereon that, when executed by the processing unit, cause the processing unit to input the extracted landmarks into the second machine learning module, where the second machine learning algorithm generates a 3D facial surface model for the subject. The memory has further computer-executable instructions stored thereon that, when executed by the processing unit, cause the processing unit to provide a correlation model between 3D facial and bone surfaces, and estimate a reference bone model for the subject using the 3D facial surface model and the correlation model.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flow chart illustrating example operations for estimating a patient-specific reference bone shape model for a patient with CMF defects according to an implementation described herein.

FIG. 2 is an example computing device.

FIG. 3 is a flow chart illustrating example operations for estimating a patient-specific reference bone shape model for a patient with CMF defects according to another implementation described herein. As shown in FIG. 3, the operations include the following steps. First, a 3D facial surface (“reconstructed 3D face”) is reconstructed from a patient's pre-traumatic 2D face photographs. Second, a correlation model between face and bone surfaces is constructed from a normal facial shape database, and an initial estimation of the patient's reference bone model is obtained from the correlation model under the input of reconstructed 3D face. Third, by deforming the initially estimated reference bone model onto the patient's current bone model (which is obtained from post-traumatic CT images), the refined reference bone model is obtained.

FIG. 4 illustrates 3D face reconstruction from 2D face photograph according to an implementation described herein.

FIG. 5 illustrates a normal facial shape database, which includes face and bone surfaces extracted from a collection of normal subjects' CT images, according to an implementation described herein.

FIG. 6 illustrates the process of searching corresponding points on bone surfaces by deforming the bone surface template onto the normal bone surfaces with the CPD matching algorithm according to an implementation described herein.

FIG. 7 illustrates the process for generating the initial reference bone model from a reconstructed face surface according to an implementation described herein.

FIG. 8 illustrates the organized neighboring vertices of the vertex Vᵢ. The red dots denote the neighboring vertices in the first layer, the green dots denote the neighboring vertices in the second layer.

FIG. 9 illustrates the CT face surface of an representative normal subject, and three pre-traumatic 2D face photographs simulated from the CT face surface.

FIG. 10 illustrates mean distance error (in mm) in terms of different setting combination of λ₁ and λ₂ in Eq. (1) for the prediction model in Eq. (2).

FIG. 11 illustrates mean distance error (in mm) in terms of the number of neighboring layers R.

FIG. 12 illustrates the qualitative evaluation results of the method illustrated in FIG. 3 applied to eight synthetic patients.

FIG. 13 illustrates the estimated bone surfaces and their surface distance heatmaps (distance between estimation and the corresponding original normal bone) obtained by four compared methods (i.e. four methods explored in the ablation studies described herein) for two randomly selected synthetic cases.

FIG. 14 illustrates the patient's normal facial bones estimated by applying the method illustrated in FIG. 3 on the real patient data, which are compared with patient's traumatic bones and post-operative bones after surgery.

FIG. 15 illustrates the patient's normal facial bones estimated by applying two alternative methods (i.e. SSM combined with ICP, sparse representation combined with TPS) and the method illustrated in FIG. 3 on the real patient data, which are compared with patient's post-operative bones after surgery.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As described above, it is difficult to simulate CMF surgical treatment for patients with complex CMF defects using the CASS platform described in Xia et al. One difficulty is due to the patient having a complex CMF defect. A complex CMF defect (e.g., result of trauma) is patient-specific, and in some cases bilateral (e.g., defects to bony structures on both sides of the face). Complex CMF defects may have sizes and/or shapes that vary from patient-to-patient. Alternatively or additionally, the location of such defects may also vary from patient-to-patient. Using the CASS platform, a patient's cephalometric measurements are compared to the cephalometric measurements of a normal patient to quantify the deformity, which is then used in the surgical simulation. While the CASS platform works well for patients with regular deformity, the CASS system is not able to quantify and restore complex CMF defects.

Estimating patient-specific normal facial bone can provide a paradigm-shift avenue in reconstructive surgery for patients with CMF defects. Techniques have been developed to estimate a patient-specific facial bone structure for a patient with CMF defects. For example, one of the most commonly used methods for patient-specific facial bone estimation is mirror-imaging mapping (see R. Schmelzeisen, et al., “Navigation-aided reconstruction of medial orbital wall and floor contour in cranio-maxillofacial reconstruction,” Injury, vol. 35, no. 10, pp. 955-962, 2004; N. C. Gellrich, et al., “Computer assisted oral and maxillofacial reconstruction,” J. Comput. Inform. Technol., vol. 14, no. 1, pp. 71-77, 2006), typically realized by mapping facial skeleton from the normal side to the defected side. Since the symmetric mapping is based on the too strict hypothesis of symmetric human facial structure, the mirror-imaging mapping technique is very limited and cannot handle the cases losing normal structures on both sides (e.g. bilateral defects).

Statistical shape model-based (SSM-based) normal facial bone estimation is another type of commonly used method. See S. Zachow, et al., “Reconstruction of Mandibular Dysplasia Using a Statistical 3D Shape Model,” in Proc. Computer Assisted Radiology and Surgery, H. Lemke et al., eds., pp. 1238-1243, 2005; W. Semper-Hogg, et al., “Virtual reconstruction of midface defects using statistical shape models,” J. Oral Maxillofac. Surg., vol. 45, no. 4, pp. 461-466, 2017; F. M. Anton, et al., “Virtual reconstruction of bilateral midfacial defects by using statistical shape modeling,” J. Oral Maxillofac. Surg., vol. 47, no. 7, pp. 1054-1059, 2019. In SSM-based methods, a set of normal facial bone shapes are first acquired from healthy subjects, upon which the principal component analysis (PCA) (see I. T. Jolliffe, Principal Component Analysis. New York: Springer-Verlag, 1986) is applied to construct an SSM (see T. Heimann and H. Meinzer, “Statistical shape models for 3D medical image segmentation: A review,” Med. Imag. Anal., vol. 13, no. 4, pp. 543-563, 2009). Then, the patient's normal bone shape is estimated by fitting the established SSM onto the remaining normal parts of patient's facial bone. A key limitation of the SSM-based methods is its weak generalization capacity due to the practical challenge that dataset available for establishing SSM is usually small.

Methods using geometric deformation have also been used to estimate normal facial bone. See L. Wang, et al., “Estimating patient-specific and anatomically correct reference model for craniomaxillofacial deformity via sparse representation,” Med. Phys., vol. 42, no. 10, pp. 5809-5816, 2015; Z. Li, et al., “Craniomaxillofacial deformity correction via sparse representation in coherent space,” in Proc. of Mach. Learning Med. Imag., 2015, pp. 69-76; S. Xie, et al., “Laplacian deformation with symmetry constraints for reconstruction of defective skulls.” in Proc. of International Conference on Computer Analysis of Images and Patterns, 2017, pp. 24-35. These methods estimate normal bone shape by deforming the patient's defected bone with the deformation field calculated by the surface interpolating techniques of thin plate spline (TPS) (see F. L. Bookstein, “Principal warps: Thin-plate splines and the decomposition of deformations,” IEEE Trans. Pattern Anal. Mach. Intell., vol. 11, no. 6, pp. 567-585, 1989) or Laplacian surface editing (see O. Sorkine, et al., “Laplacian surface editing.” in Proceedings of the Eurographics/ACM SIGGRAPH Symposium on Geometry Processing, 2004, pp. 179-188). Generally, geometric-deformation-based methods can produce accurate normal bone estimations. However, these methods cannot well handle large-scale defects, since the deformation fields quantified by using limited anatomical landmarks and surface interpolating techniques are relatively coarse.

The systems and methods described herein address the limitations of conventional systems and techniques. In conventional CMF surgical planning, for example using the CASS platform, a 3D bone model for the patient is reconstructed from a CT or CBCT scan of the patient's head. Using the CASS platform, a surgeon can simulate surgery by virtually cutting the 3D bone model into multiple pieces (e.g., 2 pieces, 3 pieces). This is referred to as performing a virtual osteotomy. The surgeon can then move the individual pieces into desired positions, for example, based on a cephalometric analysis, to correct the patient's jaw deformities. The CASS platform is described in detail in WO2018/035524, published on Feb. 22, 2018 and entitled “SYSTEMS AND METHODS FOR COMPUTER-AIDED ORTHOGNATHIC SURGICAL PLANNING.” For patient's having CMF defects, conventional surgical planning using the CASS system is not possible. While the cephalometric analysis provided by the CASS platform works well for correcting straight-forward jaw deformities, it is inadequate in treating patients with complex CMF trauma. The solution provided by the CASS platform relies on population averaged measurements, which are inadequate for planning reconstructive surgery for a patient with complex CMF defect. In these cases, a patient-specific reference anatomy is needed for planning the reconstructive surgery. Unfortunately, it is often not possible to obtain the patient's pre-trauma bone structure. For example, a patient does not typically have a 3D head scan (e.g., CT, CBCT etc. scan) taken prior to the traumatic event. It should be understood that a 3D head scan (e.g., CT, CBCT etc. scan) taken after the traumatic event has the CMF defects, i.e., it cannot be used to obtain the patient's pre-trauma bone structure. The systems and method described herein use 2D images such as portrait photographs taken prior to the traumatic event to generate a reference bone model for the patient. The reference bone model provides the patient-specific reference anatomy needed for planning of complex reconstructive surgery. The reference bone model obtained as described herein is robust and can be used to correct patient-specific CMF defects with different types and sizes and locations. The reference bone model is obtained, at least in part, using one or more machine learning algorithms. Additionally, as described below, the systems and methods described herein provide advantages over other known techniques for estimating patient-specific facial bone structure such as mirror-imaging mapping, SSM-based methods, and geometric-deformation-based methods.

Described herein are systems and methods for estimating a patient-specific reference bone shape model for a patient with CMF defects via 3D face reconstruction and a deformable shape model. This disclosure contemplates that the CMF defects may be caused by facial trauma (e.g., an accident). The systems and methods described herein use pre-traumatic 2D images (e.g., conventional portrait photographs) and post-traumatic 3D (e.g., CT scans of the patient's head). Specifically, a 3D facial surface model is first reconstructed from the patient's pre-traumatic 2D images. Thereafter, a correlation model between the facial and bone surfaces of normal subjects is constructed. In some implementations, the correlation model is constructed using a sparse representation based on the CT images of normal subjects (i.e., training data). Finally, by feeding the 3D facial surface model (which was reconstructed from the 2D images) into the correlation model, an initial patient-specific reference bone shape model is generated. Optionally, the initial reference bone shape model can be refined by applying non-rigid surface matching between the initially estimated shape and the patient's post-traumatic bone structure based on the adaptive-focus deformable shape model (AFDSM). Furthermore, a statistical shape model, built from the CT images of normal subjects (training data), can optionally be used to constrain the deformation process to avoid overfitting.

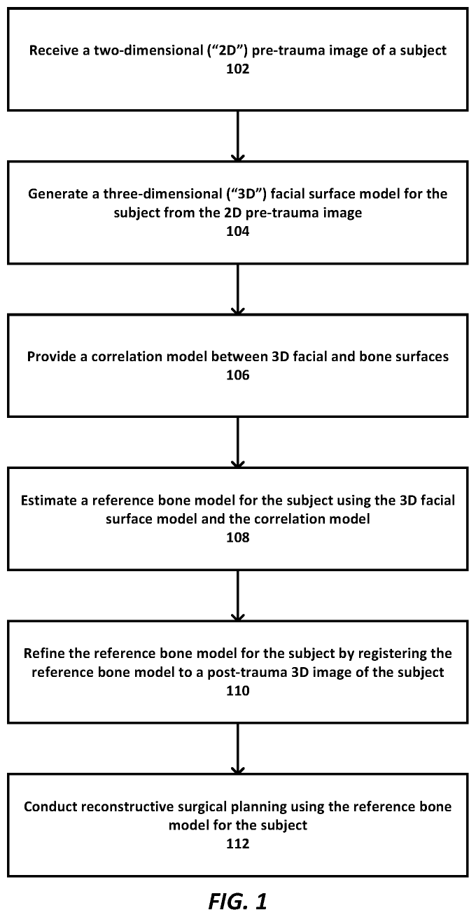

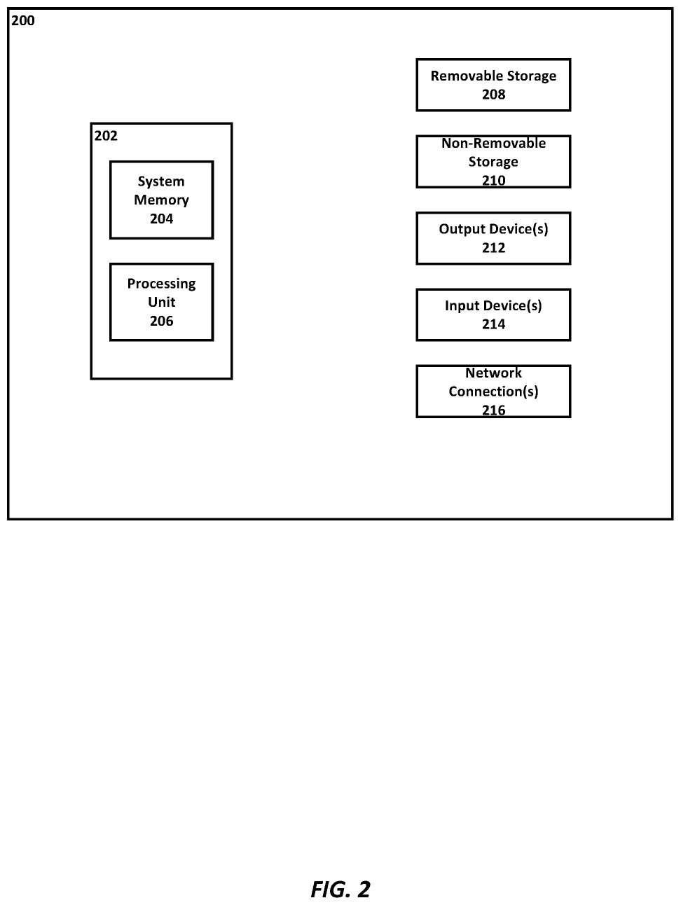

Referring now to FIG. 1, an example method for estimating a patient-specific reference bone shape model for a patient is shown. This disclosure contemplates that the operations described herein can be implemented by a computing device (e.g., computing device 200 shown in FIG. 2). As described herein, the patient has CMF defects as a result of a traumatic event such as an accident, injury, or medical procedure. For example, the patient may have sustained injury as a result of a traffic accident or combat. In other cases, the patient's CMF defects may be the result of a tumor ablation procedure or other medical procedure. CMF defects can include, but are not limited to, damage to the patient's facial bone. In some cases, the CMF defects are unilateral (e.g., only on one side of the patient's face). In other cases, the CMF defects are bilateral (e.g., on both sides of the patient's face). It should be understood that the method described herein can be used regardless of the size, shape, and/or location of CMF defects. As described herein, the goal of CMF reconstructive surgery is to reconstruct the CMF defects, for example by restoring the defected facial bone (or bones) to a normal shape, normal position, and/or desired pre-trauma status.

At step 102, a 2D pre-trauma image of a subject (also referred to herein as “patient”) is received. The 2D pre-trauma image can be a digital image. The 2D pre-trauma image includes the subject's head and facial features. For example, the 2D pre-trauma image may be a portrait photograph. This disclosure contemplates that the portrait photograph can be casual or formal. Optionally, in some implementations, a plurality of 2D pre-trauma images of the subject are received.

At step 104, a three-dimensional (“3D”) facial surface model for the subject is generated from the 2D pre-trauma image. In some implementations, there is more than one 2D pre-trauma image of the subject, and the 3D facial surface model is generated from the plurality of 2D pre-trauma images. In particular, a respective 3D facial surface model is generated from each of the 2D pre-trauma images, and thereafter all reconstructed 3D facial surface models are merged into a single 3D facial surface model (also referred to herein as “combined 3D facial surface model”). The 3D facial surface model is a 3D facial model of the subject's pre-trauma facial soft tissue. The 3D facial surface model can be generated by analyzing the 2D pre-trauma image with a machine learning algorithm. As described below, in some implementations, the machine learning algorithm is a convolutional neural network (CNN). A CNN is a type of deep neural network that has been applied to image analysis applications.

For example, step 104 can include extracting, using a first machine learning algorithm, a plurality of landmarks from the 2D pre-trauma image, and generating, using a second machine learning algorithm, the 3D facial surface model from the extracted landmarks. The first machine learning algorithm can be a CNN such as the trained CNN as described in A. Bulat and G. Tzimiropoulos, “How far are we from solving the 2D & 3D face alignment problem?(and a dataset of 230,000 3D facial landmarks),” in Proc. IEEE Int. Conf. Comp. Vis., vol. 1, no. 6, 2017, p. 8. The trained CNN described in Bulat et al. has been shown to work well for reconstructing 3D facial landmarks from 2D facial images. The second machine learning algorithm can be thin plate spline (TPS) as described in F. L. Bookstein, “Principal warps: Thin-plate splines and the decomposition of deformations,” IEEE Trans. Pattern Anal. Mach. Intell., vol. 11, no. 6, pp. 567-585, 1989.

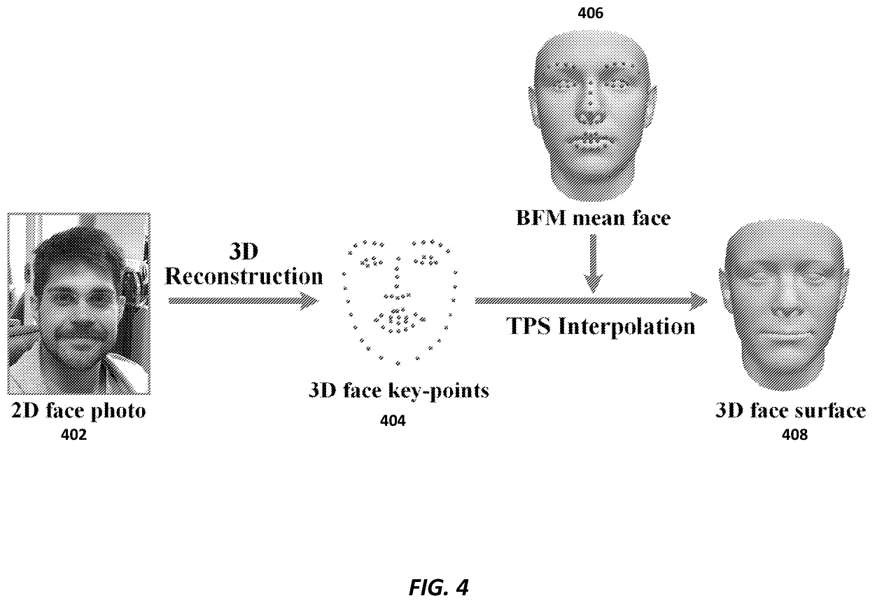

Alternatively or additionally, in some implementations and with reference to FIG. 4, step 104 of FIG. 1 includes extracting, using a CNN (e.g., the trained CNN of Bulat et al.), a plurality of landmarks (reference number 404 in FIG. 4) from the 2D pre-trauma image (reference number 402 in FIG. 4), and computing a sparse deformation field between the extracted landmarks and corresponding extracted landmarks for a reference 3D face model (reference number 406 in FIG. 4). The reference 3D face model can optionally be the Basel face model (BFM). It should be understood that BFM is provided only as an example. This disclosure contemplates using other facial surface templates as the reference 3D face model. Step 104 further includes converting the sparse deformation field into a dense deformation field (e.g., using TPS), and applying the dense deformation field on the reference 3D face model to generate the 3D facial surface model (reference number 408 in FIG. 4).

This disclosure contemplates using other machine learning algorithms including, but not limited to, a 3D morphable model (3DMM) as described in Blanz et al., Face recognition based on fitting a 3D morphable model, Pattern Analysis and Machine Intelligence, IEEE Transactions on, vol. 25, pp. 1063-1074 (2003) or deep learning techniques such as neural networks, recurrent neural networks, and/or generative adversarial networks, to generate the 3D facial surface model.

Referring again to FIG. 1, at step 106, a correlation model is provided between 3D facial and bone surfaces of normal subjects. The correlation model provides the relationship between 3D facial surfaces (e.g., soft tissue) and 3D bone surfaces of normal subjects. The correlation model is established as described below. First, a database (also referred to herein as a “normal facial shape database”) including a plurality of facial and bone surface models is created. Each of the facial and bone surface models in the database is extracted from a 3D image of a reference subject. For example, each 3D image is a 3D scan of a different subject's head, where a 3D image captures both a subject's 3D soft tissue surfaces and 3D bone structure. The 3D images can optionally be computed tomography (CT) or cone beam CT (CBCT) images. It should be understood that CT and CBCT are provided only as an example and that other imaging modalities including, but not limited to magnetic resonance imaging (MRI), may be used to capture the 3D images. Then, the correlation model is established based on the facial and bone surface models in the database. Optionally, the correlation model is established using a machine learning algorithm such as sparse representation. It should be understood that sparse representation is provided only as an example. This disclosure contemplates using other algorithms to establish the correlation model including, but not limited to, principal component analysis (PCA). In some implementations, the correlation model includes a face dictionary, which includes a matrix including a plurality of vectorized coordinates of a plurality of landmarks from each of the facial surface models. Alternatively or additionally, the correlation model includes a bone dictionary, which includes a matrix representing a plurality of vectorized coordinates of a plurality of landmarks on each of the bone surface models.

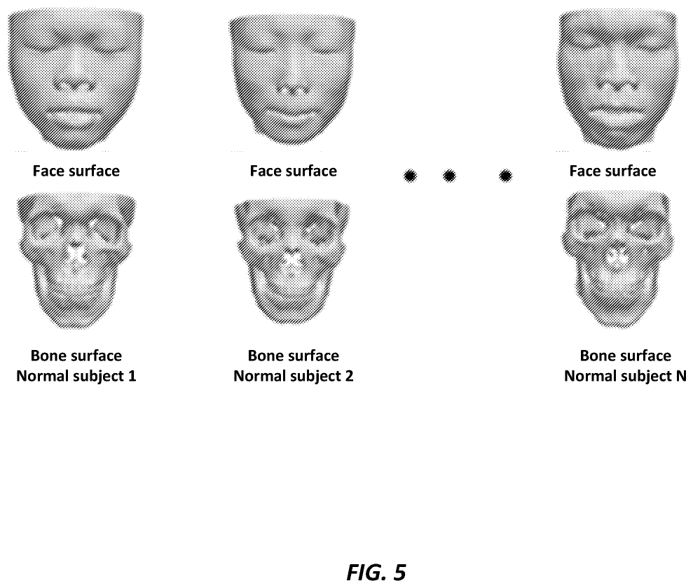

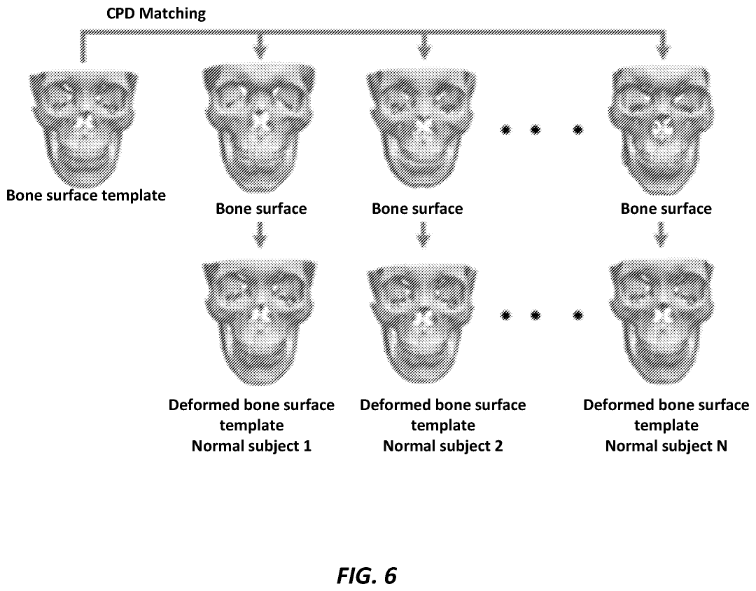

An example process for creating a normal facial shape database is described with regarding to FIG. 5. In particular, a plurality of 3D images are received, where each 3D image is a 3D scan (e.g., CT or CBCT scan) of a different subject's head. These subjects are also referred to herein as “normal subjects.” The facial soft tissue and bone structure are extracted from each of the 3D images. This disclosure contemplates using algorithms known in the art to perform segmentation on the 3D images. An example segmentation algorithm is described below in the examples. Using these segmented results, a 3D facial surface model and a 3D bone surface model are generated for each of the 3D images. The 3D facial surface models for the normal subjects are illustrated in the top row of FIG. 5, and the 3D bone surface models for the normal subjects are shown in the bottom row of FIG. 5. This disclosure contemplates using algorithms known in the art to generate 3D surfaces. An example 3D surface generation algorithm is described below in the examples. Next, a plurality of landmarks are detected on each of the 3D facial surface model and the 3D bone surface model. This disclosure contemplates using algorithms known in the art to detect landmarks. An example landmark detection algorithm is described below in the examples. Thereafter, the respective 3D facial surface models and 3D bone surface models for all of the subjects are rigidly aligned using the extracted landmarks. This disclosure contemplates using algorithms known in the art to align the surface models. An example alignment algorithm is described below in the examples. A shape template is then selected. For example, as described below in the examples, the shape template may be an aligned shape with its landmark locations closest to the average landmark locations for all aligned shapes. Finally, the template shape is deformed (e.g., non-rigidly mapped) onto each of the aligned 3D facial surface models and 3D bone surface models. This process is illustrated in FIG. 6. The facial and bone dictionaries, which are part of the correlation model, are built using correspondences between the shape template and each of the aligned 3D facial surface models and 3D bone surface models. It should be understood that that the number of normal subjects (i.e., the number of different 3D images analyzed and made part of the normal facial shape database) impacts the reliability of the correlation model. For example, if the size of the normal facial shape database is relatively large, the initial reference bone model estimate, which is estimated from the correlation model, may be more accurate. On the other hand, if the size of the normal facial shape database is relatively small, the initial reference bone model estimate may be less accurate since the new patient's facial structure is more likely to be different than those of the normal subjects.

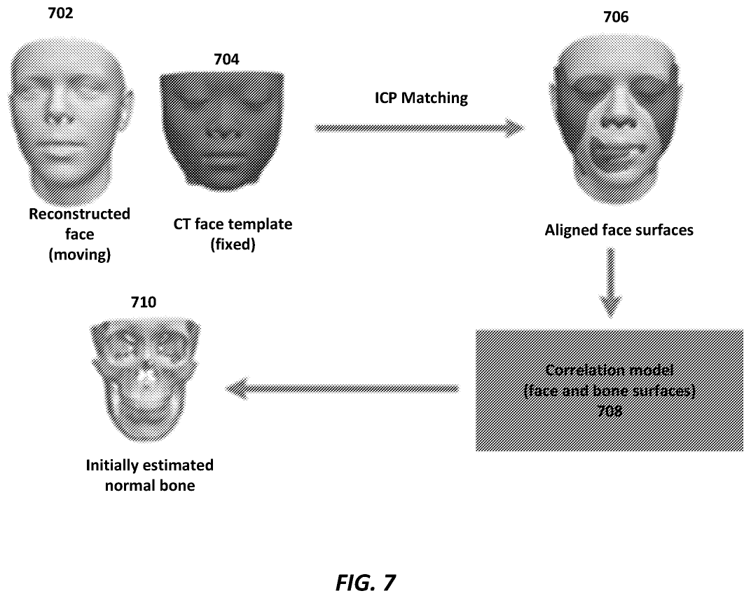

Referring again to FIG. 1, at step 108, a reference bone model (also referred to herein as “reference shape model”) for the subject is estimated using the 3D facial surface model and the correlation model. The reference bone model is a 3D model of the subject's pre-trauma facial bone structure. As described herein, the subject's pre-trauma facial bone structure is unknown. For example, a 3D scan (e.g., CT, CBCT, etc. scan) of the subject post-trauma would include the CMF defects resulting from the trauma. To perform surgical planning, it is desirable, however, to have the subject's pre-trauma facial bone structure, i.e., the 3D bone structure without CMF defects, for use as guidance during surgical planning. In some implementations and with reference to FIG. 7, step 108 of FIG. 1 includes registering the 3D facial surface model for the subject (reference number 702 in FIG. 7) and a facial image template (reference number 704 in FIG. 7). The registered 3D facial surface model is illustrated by reference number 706 in FIG. 7. This disclosure contemplates using registration algorithms known in the art such as an iterative closest point (ICP) algorithm. It should be understood that the ICP algorithm is provided only as an example and that other registration algorithms including, but not limited to, coherent point drift (CPD) or Gaussian mixture model (GMM) may be used to perform registration. Additionally, this disclosure contemplates that the facial image template can be the shape template discussed above with regard to step 106 of FIG. 1, i.e., the shape template selected while creating the normal facial shape database. The facial image template can be selected from among a plurality of facial surface models for a plurality of normal subjects. After registration, a plurality of sample points on the facial image template are mapped to the 3D facial surface model for the subject to obtain a plurality of corresponding sample points on the 3D facial surface model for the subject. This disclosure contemplates using a coherent point drift (CPD) algorithm to obtain correspondences. It should be understood that the CPD algorithm is provided only as an example and that other algorithms including, but not limited to, a non-rigid iterative closest point (ICP) algorithm may be used to obtain correspondences. Thereafter, a coordinate vector of the corresponding sample points is input into the correlation model (reference number 708 in FIG. 7) to estimate the reference bone model for the subject (reference number 710 in FIG. 7). As discussed above, the reference bone model is a 3D model of the subject's pre-trauma facial bone structure. It is patient-specific, i.e., unique to the CMF defects of the patient. The reference bone model is obtained from one or more pre-trauma 2D images of the subject. In this way, the method of FIG. 1 can be used to obtain the subject's pre-trauma facial bone structure, which is needed for planning the complex reconstructive surgery to correct CMF defects.

Referring again to FIG. 1, at step 110, the reference bone model for the subject can optionally be refined. In other words, the reference bone model is treated only as an “initial” reference bone model for the subject, which can be refined by registering the reference bone model obtained using steps 102-108 to a 3D post-trauma image of the subject. As described herein, the 3D image may be a CT or CBCT scan of the subject. The 3D post-trauma image is obtained after the traumatic event and therefore includes the CMF defects. The 3D post-trauma image of the subject can be analyzed (e.g., segmented, landmark extraction, 3D surface generation, etc.) as described herein. The reference bone model of the subject can be refined by deforming the reference bone model onto the 3D post-trauma bone model obtained from the 3D post-trauma image. Optionally, the reference bone model refinement can be constrained using a statistical shape model (SSM). Alternatively or additionally, the reference bone model can optionally be refined using a machine learning algorithm such as an adaptive-focus deformable shape model (AFDSM)

Optionally, at step 112, the reference bone model for the subject (e.g., the “initial” reference bone model obtained using steps 102-108 or the “refined” reference bone model obtained using steps 102-110) can be used for reconstructive surgical planning. As described herein, the reference bone model for the subject represents the subject's facial bone structure before the traumatic event, which resulted in CMF defects. It should be understood that steps 102-108 (or steps 102-110) of FIG. 1 facilitate estimation of the subject's pre-trauma facial bone structure from one or more pre-trauma 2D images. Reconstructive surgical planning can include, but is not limited to, simulating surgery by virtually cutting the reference bone model into multiple pieces (e.g., 2 pieces, 3 pieces) and then virtually moving the individual pieces into desired positions. The reference bone model obtained using the operations of FIG. 1 can be used by the surgeon when guiding the individual pieces into the desired positions (e.g., their pre-trauma shape and/or position). CMF reconstructive surgical planning is described in detail in WO2018/035524, published on Feb. 22, 2018 and entitled “SYSTEMS AND METHODS FOR COMPUTER-AIDED ORTHOGNATHIC SURGICAL PLANNING.” Instead of using linear and angular measurements (e.g., the cephalometric measurements) and surgeon's subjective input as described in WO2018/035524, a patient-specific bony reference shape model (e.g., the reference bone model obtained using the method described with regard to FIG. 1) can be used to accurately guide a surgeon to simulate the reconstructive surgery.

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Additionally, a neural network is a computing system including a plurality of interconnected neurons (e.g., also referred to as “nodes”). This disclosure contemplates that the nodes can be implemented using a computing device (e.g., a processing unit and memory as described with regard to FIG. 2). The nodes can optionally be arranged in a plurality of layers such as input layer, output layer, and one or more hidden layers. Each node is connected to one or more other nodes in the neural network. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the neural network, nodes in the hidden layer(s) modify the data between the input and output layers, and nodes in the output layer provide the results. Each node is configured to receive an input, implement a function (e.g., sigmoid function or rectified linear unit (ReLU) function), and provide an output in accordance with the function. Additionally, each node is associated with a respective weight. Neural networks are trained with a data set (e.g., the demonstration data described herein) to minimize the cost function, which is a measure of the neural network's performance. Training algorithms include, but are not limited to, backpropagation through time (BPTT). The training algorithm tunes the node weights and/or bias to minimize the cost function. It should be understood that any algorithm that finds the minimum of the cost function can be used to for training the neural network. Unlike a traditional neural network, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. Neural networks (including CNNs) are known in the art and therefore the network topology is not described in further detail herein.

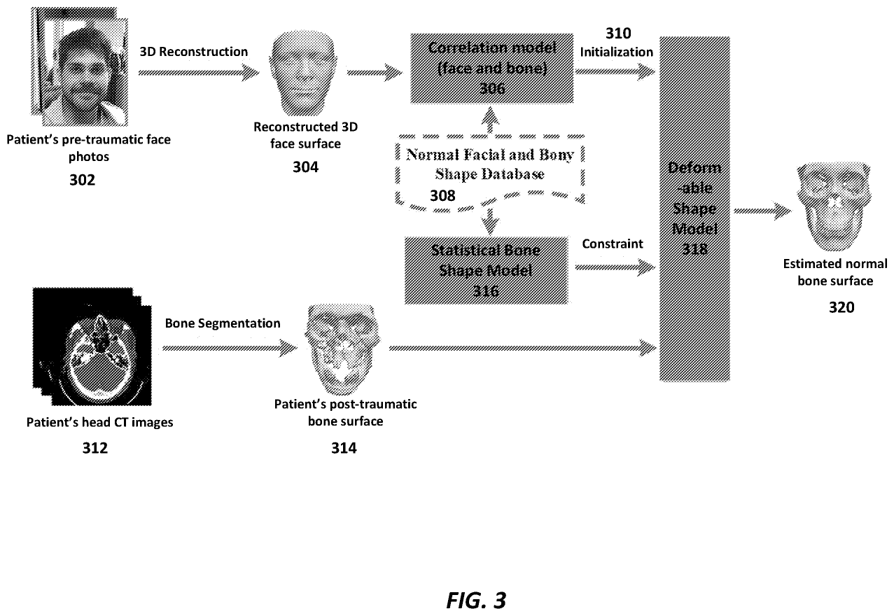

Referring now to FIG. 3, example operations for estimating a patient-specific reference bone shape model for a patient according to another implementation is shown. This disclosure contemplates that the operations of FIG. 3 can be implemented by a computing device (e.g., computing device 200 shown in FIG. 2). The method includes receiving 2D pre-trauma images 302 of a subject, and generating a 3D facial surface model 304 for the subject from the 2D pre-trauma images 302. As described herein, the 3D facial surface model 304 can be generated using one or more machine learning algorithms. The method also includes providing a correlation model 306 between 3D facial and bone surfaces of normal subjects. As described herein, the correlation model 306 can be established based on a normal facial shape database 308, which can be created from respective 3D images of a plurality of reference subjects (also referred to herein as “normal subjects”). As described herein, the correlation model 306 can be estimated using one or more machine learning algorithms. Additionally, the method also includes estimating an initial reference bone model 310 for the subject using the 3D facial surface model 304 and the correlation model 306. The initial reference bone model 310 provides the patient-specific anatomy of the subject with CMF defects. Additionally, the method further includes receiving a 3D post-trauma image 312 of the subject (e.g., CT scans). As described herein, the 3D post-trauma image 312 can be analyzed (e.g., segmented, landmarks detected, 3D bone surface generated) to generate a 3D post-trauma bone model 314. The method can further include refining the initial reference bone model 310 by deforming the initial reference bone model 310 onto the 3D post-trauma bone model 314. Additionally, the method further includes constraining the initial reference bone model refinement using a statistical shape model 316. Additionally, the initial reference bone model 310 is refined using a an adaptive-focus deformable shape model 318. The refined (or final) reference bone model is shown by reference 320 in FIG. 3. As described above with regard to FIG. 1, this disclosure contemplates using the initial reference bone model 310 or the refined reference bone model 320 during reconstructive surgical planning.

### EXAMPLES

A method for estimating a personalized reference bone shape model for surgical reconstruction of acquired CMF defects inflicted by any traumatic events is described below. The patient's pre-traumatic 2D portrait photographs can be used to estimate a reference bone model, in which the normal facial bones are retained, and the defected facial bones are restored. A set of pre-traumatic 2D face photographs (e.g., common daily life photographs) were collected for each patient. A 3D face surface for each patient was then reconstructed from these 2D photographs using a convolutional neural network (CNN)-based face reconstruction algorithm. Then, the head CT or CBCT images acquired from a set of training normal subjects were used to extract facial skins and bones, respectively, for each of the patients. Based on the extracted face and bone surfaces for normal subjects, dense correspondences among extracted surfaces (face or bone) were searched through a non-rigid surface matching algorithm. A correlation model between these two surfaces was constructed by applying sparse representation (see D. L. Donoho, “For most large underdetermined systems of linear equations the minimal l1-norm solution is also the sparsest solution,” Commun. Pure Appl. Math., vol. 59, no. 6, pp. 797-829, 2006) on the corresponding points of the extracted surfaces. Afterwards, by feeding the reconstructed 3D face (which was reconstructed from 2D photographs) into the constructed correlation model, a normal bone shape model was generated for the patient.

The generated shape model may be a relatively coarse (initial) estimation, since the 3D face reconstructed form previous photographs may not necessarily reflect the patient current face shape. It should be understood that this may lead to an inaccurate output of the correlation model. Therefore, to further improve the accuracy of the reference shape estimation, the patient's current facial bone (post-traumatic bone) was used to refine the initial estimation. The refinement was achieved by deforming the initially estimated normal bone surface onto the patient's post-traumatic bone surface with a deformable shape model. Additionally, to guarantee the normality of the estimated facial bone, the statistical shape model, which was constructed based on the facial bone surfaces extracted from the normal subjects, was used to constrain the deformable shape model applied on the estimated facial bone to avoid overfitting. After the refinement, the finally estimated reference bone shape model, which can then be applied to guiding the CMF surgical planning, was obtained.

The method described above was evaluated using both synthetic and real patient data. Experimental results show that the patient's abnormal facial bony structure can be recovered using this method, and the estimated reference shape model is considered clinically acceptable by an experienced CMF surgeon. Accordingly, this method is more suitable to the complex CMF defects for CMF reconstructive surgical planning.

Method

The overview workflow of the method described herein is illustrated in FIG. 3. The framework contains the following three parts: 1) the reconstruction of patient's normal 3D face (also referred to herein as a “3D facial surface model”), 2) estimation of initial reference shape model (also referred to herein as a “reference bone model”), and 3) refinement of the initial estimation.

Patient's Normal 3D Face Reconstruction

The patient's normal 3D face (also referred to herein as “3D facial surface model”) was estimated via 3D face reconstruction from a set of the patient's pre-traumatic 2D face photographs (see FIG. 4). Since a 2D face photograph provides limited information for accurate 3D face reconstruction, a set of 3D face key-points (also referred to herein as “landmarks”) was generated for each 2D face photograph. To achieve this, the CNN-based method proposed by Bulat et al. was used to automatically extract sixty-eight (68) 3D face key-points for each face photograph. A. Bulat and G. Tzimiropoulos, “How far are we from solving the 2D & 3D face alignment problem?(and a dataset of 230,000 3D facial landmarks),” in Proc. IEEE Int. Conf. Comp. Vis., vol. 1, no. 6, 2017, p. 8. Then, based on the detected 3D face key-points, the patient's normal face surface was derived using the mean face in Basel face model (BFM) (see P. Paysan, et al., “A 3D face model for pose and illumination invariant face recognition,” in Proceedings of IEEE International Conference on Advanced Video and Signal Based Surveillance, 2009, pp. 296-301). Specifically, for each set of 68 3D face key-points reconstructed from a single face photograph, a sparse deformation field was computed between the reconstructed key-points and the corresponding face key-points on the BFM mean face. The calculated sparse deformation field was then converted into a dense deformation field by TPS interpolation (see F. L. Bookstein, “Principal warps: Thin-plate splines and the decomposition of deformations,” IEEE Trans. Pattern Anal. Mach. Intell., vol. 11, no. 6, pp. 567-585, 1989). By applying the dense deformation field on the BFM mean face, the patient's normal face surface was estimated. As each 2D face photograph can be used to generate a normal face surface, the face reconstruction accuracy was improved by applying the method proposed in Piotraschke et al. to merge all reconstructed face surfaces into a single face surface. M. Piotraschke and V. Blanz, “Automated 3D face reconstruction from multiple images using quality measures,” in Proc. IEEE Conf. Comput. Vis. Pattern Recognit., 2016, pp. 3418-3427.

Initial Reference Bone Model Estimation

Given a reconstructed face surface (i.e., the normal 3D face or 3D facial surface model), an initial estimate of the corresponding bone surface can be made based on a correlation model between the face and bone surfaces, under the assumption that these two surfaces are highly correlated. Specifically, a normal facial shape database, consisting of face and bone surfaces extracted from a set of head CT scans for normal subjects (see FIG. 5), was constructed. Then, the sparse representation technique (see D. L. Donoho, “For most large underdetermined systems of linear equations the minimal l1-norm solution is also the sparsest solution,” Commun. Pure Appl. Math., vol. 59, no. 6, pp. 797-829, 2006) was used to estimate the reconstructed face surface of a patient with the normal face surfaces in the database. Finally, by applying the learned sparse coefficients on the corresponding normal bone surfaces, the patient's normal bone structure was estimated. The detailed steps in obtaining the initial bone surface estimate are described as follows.

A normal facial shape database was constructed. The normal facial shape database consists of face and bone surfaces extracted from a set of CT scans of normal subjects. Specifically, using methods described by Wang et al. (see L. Wang, et al., “Automated bone segmentation from dental CBCT images using patch-based sparse representation and convex optimization,” Med. Phys., vol. 41, no. 4, 2014; L. Wang, et al., “Automated segmentation of dental CBCT image with prior-guided sequential random forests,” Med. Phys., vol. 43, no. 1, pp. 336-346, 2016), the facial soft tissue and bone structure for the head CT scan of each normal subject were first segmented. Then, based on the segmented results, both the face and bone 3D surface models were generated by using the marching cubes algorithm (see W. E. Lorensen, and H. E. Cline, “Marching cubes: A high resolution 3D surface construction algorithm,” Computer Graphics, vol. 21, no. 4, pp. 163-169, 1987). The generated (face and bone) surfaces from different subjects were then properly aligned for subsequent correlation model. That is, the method described by Zhang et al. (see J. Zhang, et al., “Automatic craniomaxillofacial landmark digitization via segmentation-guided partially-joint regression forest model and multiscale statistical features,” IEEE Trans. Biomed. Eng., vol. 63, no. 9, pp. 1820-1829, September 2016) was first used to extract 51 anatomical landmarks on each facial bone structure, and these landmarks were then used to rigidly align all extracted shapes (i.e., face and bone surfaces) from different subjects together (see FIG. 5). Furthermore, to establish dense correspondences for the surface points on the extracted shapes across different subjects, a template shape was non-rigidly mapped onto each aligned shape using coherent point drift (CPD) algorithm (see A. Myronenko, and X. Song, “Point set registration: Coherent point drift,” IEEE Trans. Pattern Anal. Mach. Intell., vol. 32, no. 12, pp. 2262-2275, 2010) (see FIG. 6). The template shape (also referred to herein as “facial image template”) was defined as the aligned shape that has its landmark location closest to the average bone landmark locations from all the aligned shapes. Once the dense correspondences between the template and aligned shapes were established, two dictionaries (i.e., one for the face surface and one for the bone surface) were built containing all the corresponding surface points from all normal subjects.

A correlation model was constructed. Based on the assumption of linear correlation between the face and bone surfaces, the sparse representation technique was applied to establish a correlation model between them. To this end, based on the surface corresponding points (between the template and aligned shapes, as described in the previous step) for all normal subjects in the facial shape database, two dictionaries, i.e., the face and bone dictionaries, were constructed and then utilized in the correlation model. Specifically, the coordinates of all the corresponding points on the aligned face surfaces were combined into a 3M×N matrix as the face dictionary D_{Face}, where M is the number of corresponding points on each face surface (M=36,213), and N is the number of normal subjects. Each column of the matrix corresponds to the (vectorized) coordinates of M corresponding points of one subject from the database. As three values are used to define the coordinate of a point in the 3D space, the number of elements in each column is 3M. Similarly, the coordinates of all the corresponding points (30,812 points) on the aligned bone surfaces in the database were stacked together into a matrix as the bone dictionary D_{Bone}. Then, given a coordinate vector of the corresponding points on a patient's estimated normal face surface, P_{Est}^{F} (which will be detailed in the following step), a sparse representation model was used to sparsely represent it with the column vectors in the face dictionary D_{Face}. The sparse coefficient vector C was calculated by solving the following objective shown by Eq. (1):

$${C}_{min} = arg {min}_{C} {‖ {D}_{Face} C - {P}_{Est}^{F} ‖}_{2}^{2} + {λ}_{1} {‖ C ‖}_{1} + {λ}_{2} {‖ C ‖}_{2} , \qquad ( 1 )$$

where the first term is the data fitting term, ∥•∥₂ denotes the l2 norm, ∥•∥₁ denotes the l1 norm, and λ₁ and λ₂ are the two regularization parameters used to control the sparsity of C. Eq. (1) is similar to the well-studied Elastic Net problem (see H. Zou and T. Hastie, “Regularization and variable selection via the elastic net,” Journal of the Roy. Statist. Soc., ser. B, vol. 67, no. 2, pp. 301-320, 2005), which can be solved using the method proposed in Zou et al. With the calculated sparse coefficient vector Cₘᵢₙ, the patient's normal bone surface points P_{Est}^{B} were estimated by Eq. (2):

P_{Est}^{B}=D_{Bone}Cₘᵢₙ, (2)

under the assumption that the face and respective bone surfaces are highly correlated. Then, the patient's normal bone surface model can be derived from the bone template surface mesh based on the estimated points.

The initial normal bone shape can then be estimated. To use the correlation model described in the previous step, the correspondence between the normal face estimated from 2D photographs (e.g., the 3D facial surface model described above) and the CT face template are first found. As the estimated normal face and the CT face template were acquired from different imaging spaces, the estimated face was mapped onto the CT face template image space using the iterative closest point (ICP) algorithm (see P. J. Besl and N. D. McKay, “A method for registration of 3D shapes,” IEEE Trans. Pattern Anal. Mach. Intell., vol. 14, pp. 239-256, February 1992) with a similarity transform (rigid and scale transform). Then, non-rigid surface matching between the mapped face and the CT face template was then performed using the CPD algorithm, where, for each sampled point on the CT face template, the corresponding point on the mapped face was obtained. Finally, by inputting the coordinate vector of the corresponding points (i.e., P_{Est}^{F}) into the established correlation model (i.e., Eq. (1) and Eq. (2)), an initial normal bone shape (e.g., the reference bone model) for the patient was obtained. The whole procedure is illustrated in FIG. 7.

Estimation Refinement

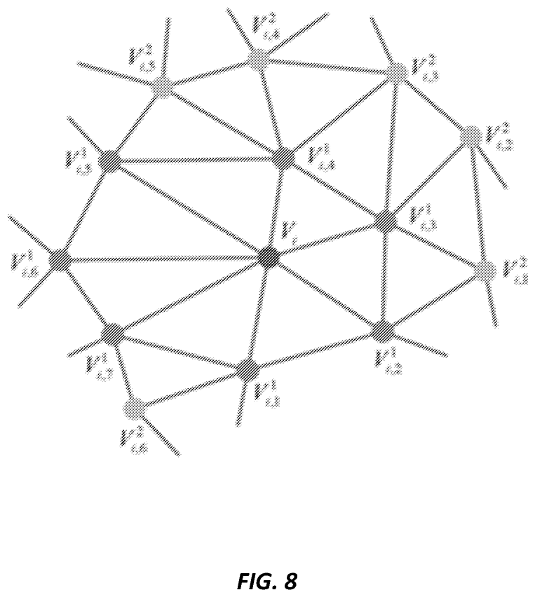

To refine the initially estimated reference bone shape, the adaptive-focus deformable shape model (AFDSM) (see D. Shen, and C. Davatzikos, “An adaptive-focus deformable model using statistical and geometric information,” IEEE Trans. Pattern Anal. Mach. Intell., vol. 22, no. 8, pp. 906-913, 2000), a variant of snake model (see M. Kass, et al., “Snakes: Active contour models,” Int. J. Comput. Vis., vol. 1, pp. 321-331, 1988), was used to deform the initial estimation onto the patient's post-traumatic facial bone. The AFDSM-based non-rigid surface matching was realized by first defining an attribute vector on each vertex, so that the vertex differences before and after the deformation could be computed. To construct the attribute vector, the neighboring vertices of each vertex Vᵢ were first organized into different layers on the surface mesh, where each neighboring vertex V_{i,j}^{k} in the k^{th} layer was connected to Vᵢ by k edges. FIG. 8 illustrates the organized neighbors of a vertex on a surface mesh. Then, the attribute vector Fᵢ of vertex Vᵢ was defined as Eq. (3):

$${F}_{i} = \frac{{[ \begin{matrix}{f}_{i , 1} & {f}_{i , 2} & {f}_{i , 3} & … & {f}_{i , R}\end{matrix} ]}^{T}}{{\sum }_{i = 1}^{N} {\sum }_{k = 1}^{R} \vert {f}_{i , k} \vert} , \qquad ( 3 )$$

$${f}_{i , k} = \vert \begin{matrix}{x}_{i} & {x}_{i , 1}^{k} & {x}_{i , 2}^{k} & {x}_{i , 3}^{k} \\ {y}_{i} & {y}_{i , 1}^{k} & {y}_{i , 2}^{k} & {y}_{i , 3}^{k} \\ {z}_{i} & {z}_{i , 1}^{k} & {z}_{i , 2}^{k} & {z}_{i , 3}^{k} \\ 1 & 1 & 1 & 1\end{matrix} \vert ,$$

where f_{i,k} denotes the determinant of a 4×4 matrix which contains position information of vertex Vᵢ (i.e., [xᵢ yᵢ zᵢ]) and its three nearest neighbors (i.e., {[x_{i,j}^{k}, y_{i,j}^{k}, z_{i,j}^{k}], j=1, 2, 3}) within the k^{th} layer, R denotes the number of neighboring layers for Vᵢ, and N is the total number of vertices on the surface mesh. Generally, Fᵢ reflects the local shape information when R is small, and gradually captures more global shape information as R increases. Based on the attribute vector Fᵢ, the energy function was defined as Eq. (4):

E=Σᵢ₌₁^{N}(Eᵢ^{model}+Eᵢ^{data}), (4)

where Eᵢ^{model} denotes the degree of attribute vector difference between the initial and its deformed version for vertex Vᵢ, and Eᵢ^{data} denotes the degree of attribute vector difference between the deformed and target shapes for vertex Vᵢ. The initial and target shapes were defined as the initially estimated bone surface and the patient's post-traumatic bone surface, respectively. In other words, by minimizing energy in Eq. (4), it is ensured that the refined normal bone surface did not deviate too much from both the initial estimated and patient's post-traumatic bone surfaces. A greedy deformation algorithm (see D. Williams and M. Shah, “A fast algorithm for active contours and curvature estimation,” Comput. Vis. Graph. Image Process., vol. 55, no. 1, pp. 14-26, 1992) was applied to minimizing the energy function E in Eq. (4) based on affine transform.

Furthermore, to guarantee the normality of the deformed shape after each optimization iteration, the deformation was also constrained to be within a statistical normal shape. Specifically, a statistical shape model (SSM) (see T. Heimann and H. Meinzer, “Statistical shape models for 3D medical image segmentation: A review,” Med. Imag. Anal., vol. 13, no. 4, pp. 543-563, 2009) of the facial bone shape from the normal facial shape database was constructed via PCA (see I. T. Jolliffe, Principal Component Analysis. New York: Springer-Verlag, 1986), such as shown by Eq. (5):

S_{SSM}ⁱ=S̅+WⁱP, (5)

where S_{SSM}ⁱ denotes the reconstructed bone shape by applying the SSM on Sⁱ, Sⁱ denotes the deformed bone shape at the i^{th} iteration, S̅ denotes the mean aligned bone shape in the normal facial bone shape database, Wⁱ is a coefficients vector, and P is a matrix of principal components of normal bone shape. Here, each shape is represented as a vector of coordinates of all vertices on a bone surface model. The constructed SSM was then used to constrain each iteration of the optimization to avoid deformation overfitting, such as shown by Eq. (6):

S_{updated}ⁱ=αᵢS_{SSM}ⁱ+(1−αᵢ)Sⁱ, (6)

where S_{updated}ⁱ is the correspondingly updated bone shape, and αᵢ is a hyperparameter between zero and one that determines the weight between S_{SSM}ⁱ and Sⁱ in the i^{th} iteration. αᵢ was gradually reduced at each iteration so that the last deformed shape is closer to the target shape. At each iteration, the deformed shape was updated and then used as input for the next iteration.

Results

Experimental Dataset

A set of CT scans of 30 normal subjects were used in this study. These CT scans were obtained from a digital archive (see J. Yan, et al., “Three-dimensional CT measurement for the craniomaxillofacial structure of normal occlusion adults in Jiangsu, Zhejiang and Shanghai Area,” China J. Oral Maxillofac. Surg., vol. 8, pp. 2-9, 2010) at the Department of Oral and Craniomaxillofacial Surgery of Shanghai Ninth People's Hospital, Shanghai, China. All these data were de-identified in accordance with the Health Insurance Portability and Accountability Act (HIPAA). The image resolution of these images is 0.49×0.49×1.25 mm³, and the image size varies from 512×512×196 to 512×512×236. Additionally, image segmentation and landmark digitization was performed on the CT scans according to the methods described herein. After that, a total of 51 anatomical landmarks were localized on the facial bone for each subject.

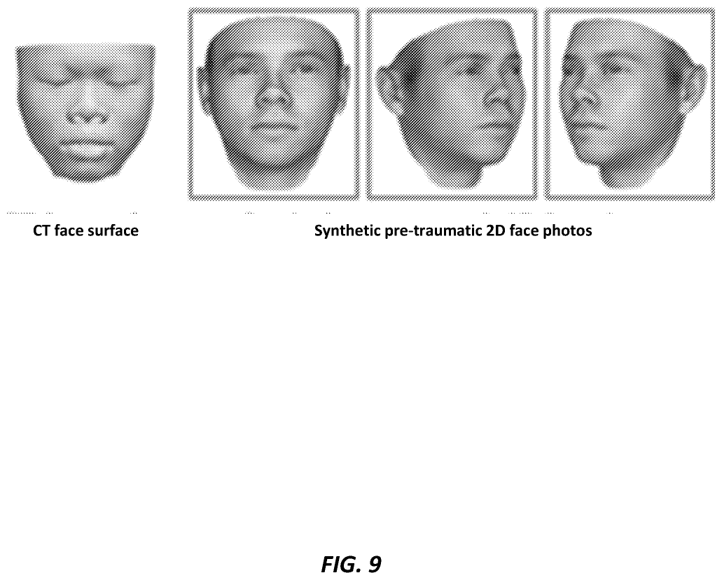

The experimental data were split into two parts: synthetic patient data and real patient data. The synthetic patient data were generated from CT images of normal subjects. To simulate pre-traumatic 2D face photographs, the BFM mean face surface was first deformed onto a normal face CT surface using the CPD algorithm, and then a statistical face texture model (see P. Paysan, et al., “A 3D face model for pose and illumination invariant face recognition,” in Proceedings of IEEE International Conference on Advanced Video and Signal Based Surveillance, 2009, pp. 296-301) was applied on the deformed face surface to set a color value on each surface vertex. Afterwards, the deformed face surface with the skin texture was rendered in 3D space for visualization and screen shots of the displayed 3D face were taken with different view degrees to generate 2D face photographs (see FIG. 9). The synthetic post-traumatic bone shape was generated by manually modifying a normal CT bone structure. An experienced CMF surgeon was recruited to remove and deform bony segments on a normal CT bone structure to simulate acquired CMF defects. The simulation was performed with an open source toolkit for editing surface meshes from the BLENDER FOUNDATION of Amsterdam, Netherlands. Specially, by using the toolkit, the surgeon removed a part of structure from the original normal facial bone, and mimicked the deformities around fracture edges of the remained part. In this way, a total of 30 simulated bones with different defect types were obtained. For the real patient data, CT scans were collected from 13 patients who suffered from severe CMF defects acquired by trauma. Additionally, the patients' two or three face photographs acquired before the trauma were also collected. Image segmentation and bone landmark digitization was also performed on real patient CT images, as were also done on CT scans of normal subjects.

Parameter Optimization

The performance of the method described herein is affected by parameters from three main component parts of the framework described above. Parameters for each component are analyzed and optimized as follows.

For the 3D face surface reconstruction (e.g., the 3D facial surface model), of which the parameters are involved in the 3D face key-points reconstruction algorithm and the number of face photographs utilized. Because the algorithm from Bulat et al. is able to produce state-of-the-art performance on 3D face key-points reconstruction, the trained model as well as its default model parameters from Bulat et al. A. Bulat and G. Tzimiropoulos, “How far are we from solving the 2D & 3D face alignment problem?(and a dataset of 230,000 3D facial landmarks),” in Proc. IEEE Int. Conf. Comp. Vis., vol. 1, no. 6, 2017, p. 8 were applied. The face key-points can be reconstructed from a single face photograph, thus an accurate normal 3D face surface can be generated from at least one of the patient's pre-traumatic face photographs with the frontal view. Since the 3D face surfaces fusion algorithm (see M. Piotraschke and V. Blanz, “Automated 3D face reconstruction from multiple images using quality measures,” in Proc. IEEE Conf. Comput. Vis. Pattern Recognit., 2016, pp. 3418-3427) used, more face photographs would result in a better 3D face reconstruction.

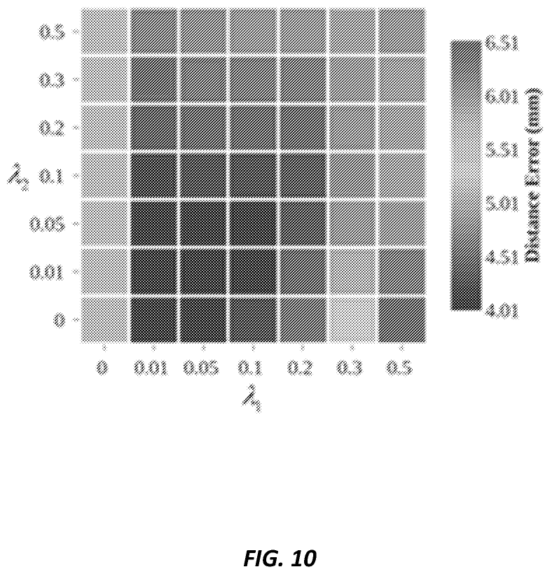

To get the correspondence between facial shapes of different normal subjects, the CPD algorithm with default parameters from its original version introduced in A. Myronenko, and X. Song, “Point set registration: Coherent point drift,” IEEE Trans. Pattern Anal. Mach. Intell., vol. 32, no. 12, pp. 2262-2275, 2010 was used. Since the structures of facial shapes (bone and face surfaces) are similar across different subjects in the normal subject database, the CPD algorithm with default parameters can result in a well surface matching performance with the matching error of less than 1 mm. To determine the parameter values of sparse representation in Eq. (1), leave-one-out cross validation was performed on CT scans of 30 normal subjects. For each leave-one-out test, one subject was selected as the testing sample, while the remaining 29 subjects were used to construct the dictionaries. The estimation performance of sparse representation in Eq. (1) and Eq. (2) was evaluated via measuring the distance error between the corresponding landmarks on the estimated and original normal bone structures. An average distance error from a total of 30 tests was calculated for each setting combination of λ₁ and λ₂. The errors in terms of applying different settings of λ₁ and λ₂ are shown in FIG. 10. It can be observed that there is no significant difference on the errors when λ₁, λ₂ϵ[0.01, 0.3], indicating that the method described herein is relatively robust to the values of λ₁ and λ₂. In following experiments, the parameters λ₁ and λ₂ were set as 0.1 and 0.01, respectively.

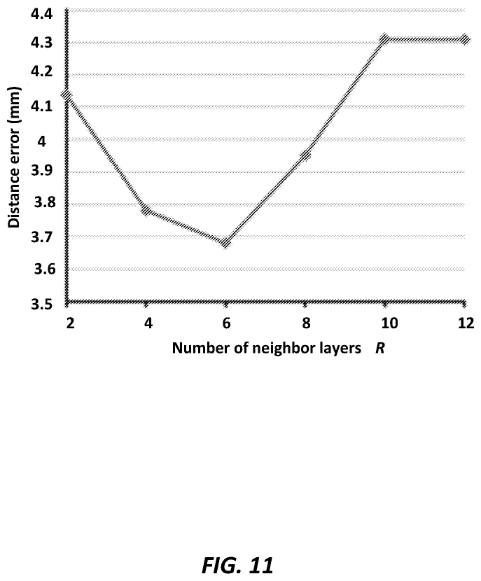

Furthermore, the parameter values for AFDSM were determined via evaluating the method described herein on synthetic patient data. For each parameter setting, a distance error was computed by comparing the landmarks on the final estimated facial bone and the corresponding landmarks on the original normal facial bone. To determine the number of neighboring layers R, the distance errors were calculated using different values of R, as shown in FIG. 11. From FIG. 11, it can be seen that the optimal values of R span in the range from 4 to 8. R was fixed as 6 in the following experiments. The shape weight coefficient αᵢ (see Eq. (6)) is used to balance the surface deformation from AFDSM and the shape constraint (to be normal) from SSM. A dynamic setting strategy was applied for assigning values on the coefficient αᵢ along with increasing steps of the deformation iterations. Specially, a large value was assigned on αᵢ for SSM to rectify the deformed bone to be normal in the early stage of the iteration, because the magnitude of deformation from AFDSM is relatively large during this stage, and the large deformation would warp initially estimated bone to be abnormal. With the increasing of iteration steps, the value of αᵢ was reduced gradually to release the constraint on the deformed bone. Once the deformation is completed after many iteration steps, the final estimated bone, which is close to the patient's original pre-traumatic facial structure and also looks like normal, was obtained. Similarly, the shape weight αᵢ was also optimized, and the maximum and minimum value of shape weight were set as 0.95 and 0.6, respectively.

Evaluation of Synthetic Dataset

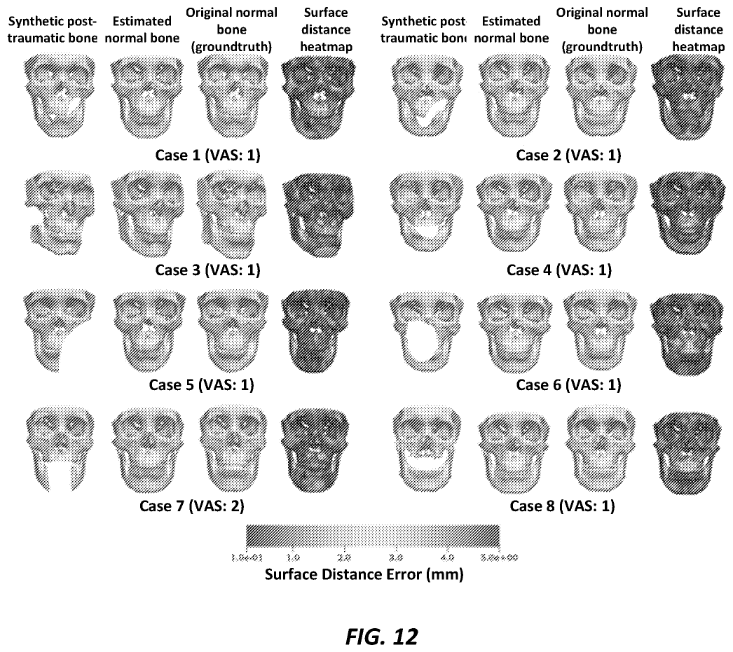

With the optimal parameters setting, the method described herein was evaluated on the synthetic patient data via leave-one-out method. For each leave-one-out experiment, one normal subject was selected as the testing sample, while the remaining 29 normal subjects were used to construct a normal facial shape database. For each testing sample, the synthetic patient data as described above was generated. The synthetic patient data was then fed into the framework described herein to estimate the reference facial bone shape of the testing sample. The qualitative evaluation was completed by a different CMF surgeon, who ranked the similarity of the two surfaces using a 1-3 visual analog score (VAS), 1: the same, 2: similar but not the same, and 3: different. FIG. 12 shows the qualitative evaluation results for eight randomly selected testing subjects, where the simulated post-traumatic, estimated reference, original normal bone surfaces, and surface distance heatmap between the estimation and original normal bone for each subject are shown in each row. The qualitative results of synthetic data also showed: same ( 26/30), similar ( 4/30), and different ( 0/30). For each subject, the performance of the method was also quantitatively evaluated by measuring an average distance error between the corresponding landmarks on the estimated and original facial bone surfaces. Results of quantitative evaluation for all cases are summarized in Table I. All these results show that the estimated reference bone shape is similar with the corresponding original normal bone shape. Thus, the defective facial bone with different types of defects can be recovered to the normal looking shape using the method described herein.

TABLE I
Facial Bone Estimation Error (in mm) of the Reference
Bone Model Method using the Synthetic Data

| | Standard | | | |
|---|---|---|---|---|
| Mean | Deviation | Median | Minimum | Maximum |
| 3.68 | 0.43 | 3.66 | 2.91 | 4.90 |

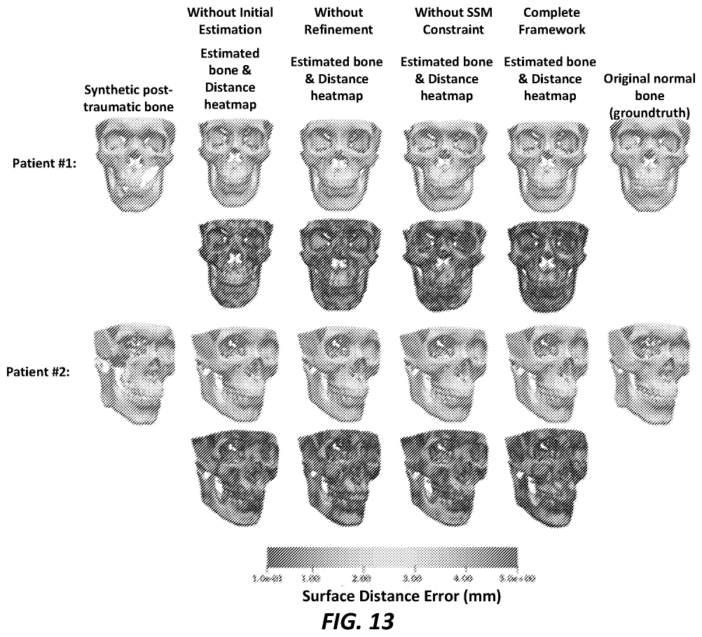

Ablation studies were also performed by removing a component part from the framework described herein one at a time, and keeping the remaining parts as an alternative method for comparison. This study demonstrated the importance of each component part in obtaining accurate results using the framework. Regarding three main component parts in the framework: 1) initial estimation from 2D face photographs; 2) refinement for initial estimation; and 3) SSM constraint in the AFDSM applied for refinement, the corresponding components were removed from the framework to get three different comparison methods, respectively. Based on the generated synthetic dataset, leave-one-out evaluation was conducted for four comparison methods, which are marked as the method without initial estimation (i.e., missing main component (1)), the method without refinement (i.e., missing main component (2)), the method without SSM constraint (i.e., missing main component (3)), and the complete version of framework (e.g., the method illustrated by FIG. 3), respectively. Estimation accuracy of the four methods were calculated as the mean distance between corresponding landmarks on estimated bone surface and the original normal bone (groundtruth). Estimation results were presented along with distance heatmaps on two representative cases selected from 30 tests for four comparison methods in FIG. 13. The quantitative results including the mean distance error and standard deviation are summarized in Table II. Paired t-test on calculated distance errors were also performed to check whether or not there is significant difference on accuracy between three comparison methods and the complete framework. From both qualitative and quantitative results calculated for the four methods, it can be observed that the method with complete framework achieved significantly better performance than all three alternative methods (p<0.05), that is including all three main component parts in the framework yields a better estimation.

TABLE II
Average Facial Bone Estimation Errors
(in mm) of Four Compared Methods Explored
in our Ablation Studies using the Synthetic Data

| Without | | Without | |
|---|---|---|---|
| initial | Without | SSM | Complete |
| estimation | refinement | constraint | framework |
| 5.51 ± 1.63 | 4.02 ± 0.43 | 5.61 ± 1.82 | 3.68 ± 0.43 |

Evaluation on Real Dataset

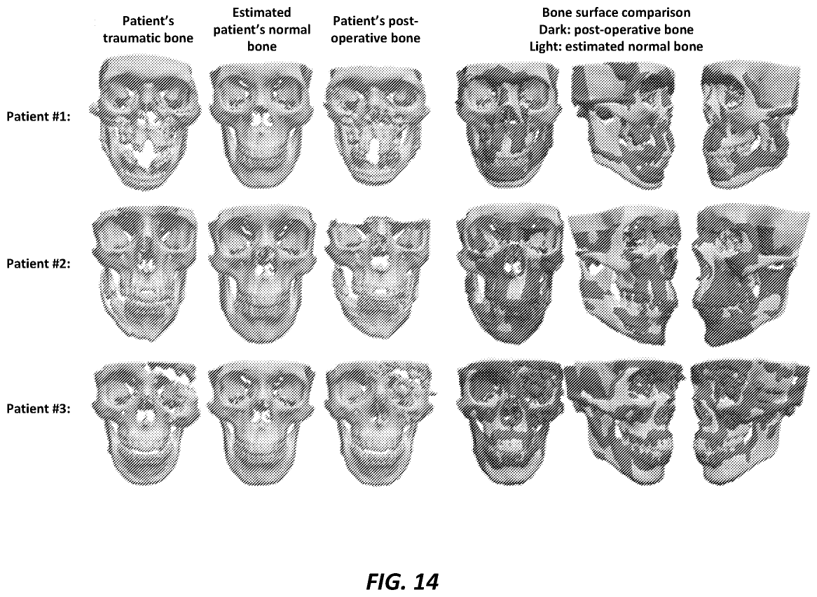

The framework described herein was also evaluated using the real patient data. The patient's original traumatic (pre-operative) bone shape, the estimated reference bone shape, and post-operative bone shape are shown in FIG. 14. Here the post-operative bone yielded by the surgery was planned using conventional CASS method described, for example, in Xia et al. The estimation was also compared with post-operative bone shape in FIG. 14. According to an experienced CMF surgeon after visual inspection of the results, the estimated bone shape obtained using the method described herein was clinically acceptable.

For quantitative evaluation of the method, an evaluation metric called Recovery Degree (RD), which is a product of two measurements, i.e., shown by Eq. (7) was defined:

RD=SD×ND, (7)

where SD denotes the Similarity Degree that computes the similarity between the estimated and post-traumatic facial bones at the normal regions of the patient's post-traumatic bone, while ND denotes the Normality Degree that computes the similarity between the estimated and the statistical normal facial bone. The SD and ND are mathematically defined as shown by Eq. (8) and Eq. (9), respectively:

$$S D = \frac{1}{D i s ( {L}_{Pat}^{normal} , {L}_{Est}^{normal} ) + 1} , \qquad ( 8 )$$

$$ND = \frac{1}{D i s ( {L}_{Recon} , {L}_{Est} ) + 1} , \qquad ( 9 )$$

where Dis(•) denotes the average Euclidean distance between two sets of corresponding bone landmarks. L_{Pat}^{normal} denotes the landmarks on the normal part of the patient's post-traumatic facial bone structure, where the normal part is identified by an experienced CMF surgeon. L_{Est}^{normal} denotes the landmarks on the estimated facial bone surface corresponding to L_{Pat}^{normal}, L_{Recon} denotes the landmarks on the statistically normal facial bone that were generated by reconstructing the estimated facial bone landmarks (i.e., L_{Est}) using principle components of the normal subjects' landmarks. Specifically, PCA was first performed on a matrix that contains all the facial bone landmarks of 30 normal subjects, where each column of the matrix consists of all the vectorized landmark locations of a subject. After that, a set of principal components of landmark locations, which were subsequently used to reconstruct L_{Est} via sparse regression, were obtained. The set of reconstructed landmarks using this statistical method is denoted as L_{Recon}. Up to this point, SD and ND in Eq. (8) and Eq. (9) are not properly scaled, and thus their values might be skewed and not very useful in making comparison. Therefore, Dis(•) in Eq. (8) and Eq. (9) was normalized as shown by Eq. (10):

$$= \frac{Dis ( \cdot ) - {Dis}_{min}}{D i {s}_{max} - D i {s}_{min}} , \qquad ( 10 )$$

where [symbol] means the normalized Dis(•), while Disₘᵢₙ and Disₘₐₓ denote the minimum and the maximum of Euclidean distance errors obtained from bone landmarks on normal subjects, respectively. Specifically, for SD, its Disₘᵢₙ and Disₘₐₓ were set as the minimum and maximum values of 30 distance errors obtained in the quantitative evaluation of the method on the synthetic patient data, respectively (i.e., Disₘᵢₙ^{SD}=2.910, Disₘₐₓ^{SD}=4.900). Similarly, for ND, the minimum and maximum values were determined from another set of distance errors. The distance errors were measured on the bone landmarks of each of 30 normal subjects by using the same approach applied on Dis(L_{Recon}, L_{Est}) computation (i.e., Disₘᵢₙ^{ND=1.989}, Disₘₐₓ^{ND}=3.452).

To demonstrate that the method described herein is superior when compared with relevant methods, the method with two alternative methods derived from related works, i.e., an SSM-based method and sparse representation method.

One alternative method is based on the SSM followed by the refinement with ICP algorithm, which is similar to the approach used in F. M. Anton, et al., “Virtual reconstruction of bilateral midfacial defects by using statistical shape modeling,” J. Oral Maxillofac. Surg., vol. 47, no. 7, pp. 1054-1059, 2019. Specially, landmarks on unaffected region of the patient's post-traumatic bone were selected, and the selected landmarks were represented using a SSM established from the landmarks on training normal bones. A set of PCA coefficients for the selected landmarks were then obtained. Based on the representation from SSM, all training normal bone surfaces weighted with the calculated PCA coefficients were merged to generate an initial estimation of normal bone. After that, the initial estimation was refined through deforming the estimated normal bone onto the patient's post-traumatic bone by ICP algorithm based on affine transform.

The other alternative method is derived from the spare representation method used in L. Wang, et al., “Estimating patient-specific and anatomically correct reference model for craniomaxillofacial deformity via sparse representation,” Med. Phys., vol. 42, no. 10, pp. 5809-5816, 2015]. In this method, the landmarks were selected from unaffected region of the patient's post-traumatic bone, and the normal landmarks on the remained affected region were estimated by sparse representation as that in L. Wang, et al., “Estimating patient-specific and anatomically correct reference model for craniomaxillofacial deformity via sparse representation,” Med. Phys., vol. 42, no. 10, pp. 5809-5816, 2015. By combining the landmarks on the unaffected region and the estimated normal landmarks on the affected region, the complete landmarks of the expecting normal bone were obtained, and the normal bone surface was then generated from the normal bone template using the TPS technique applied on the combined bone landmarks and the corresponding landmarks from bone template.

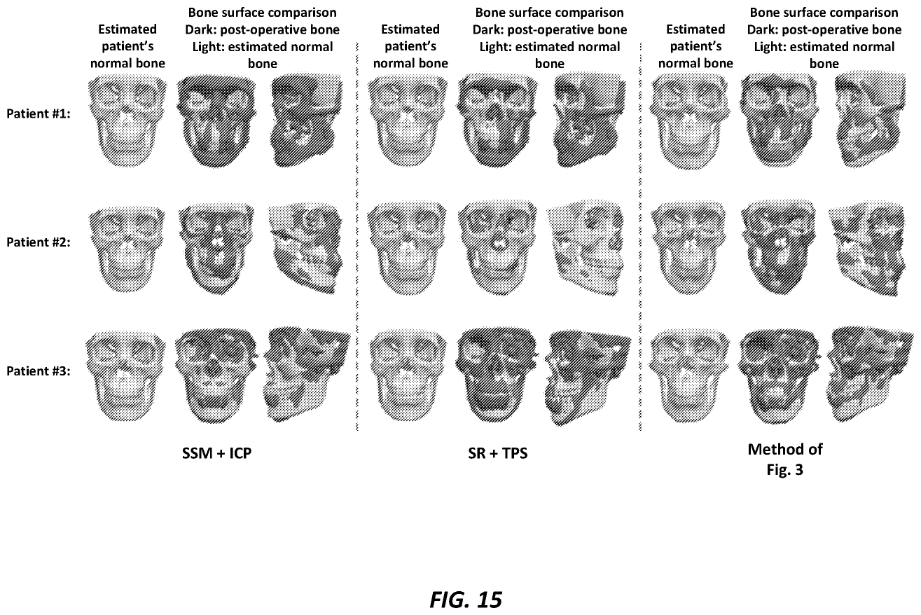

The two alternative methods along with the method described herein (e.g., the method illustrated by FIG. 3) were evaluated on real patient dataset, and the results were summarized in FIG. 15 and Table III, respectively. From the FIG. 15, the normal bones estimated from the method described herein are closely fitted with the patient's post-operative bone surfaces, while the two alternative methods bring the worse fitting performance compared with the method described herein. In Table III, the method described herein achieved higher recover degree than the other two methods (p<0.05), which also indicates that the framework described herein is superior relative to alternative methods for estimating the reference bony structure.

TABLE III
Statistics of Recovery Degree (RD) for
Estimated Reference Shape Models
Generated by Three Competing Methods

| | | Standard | | | |
|---|---|---|---|---|---|
| | Mean | Deviation | Median | Minimum | Maximum |
| SSM + ICP | 0.57 | 0.16 | 0.60 | 0.27 | 0.79 |
| SR + TPS | 0.65 | 0.23 | 0.65 | 0.33 | 0.99 |
| Proposed | 0.75 | 0.22 | 0.70 | 0.41 | 0.99 |
| method | | | | | |

Computational Cost

The computational cost for the method described herein was also summarized. The time cost for applying the proposed model on one testing subject is influenced by the number of vertices on surface meshes, as well as the iteration number for AFDSM algorithm. To accelerate the method, the surface meshes were simplified by reducing the number of surface vertices to be 5 percent of the original size. The maximum iteration number of AFDSM algorithm was set to 400 empirically. With an Intel i7-7700K CPU of 4.20 GHz and 64 GB random-access-memory, the total processing time for a new testing subject was about 4 minutes, including 1 second for 3D face reconstruction, 36 seconds for initial reference shape model estimation, and 200 seconds for shape refinement.

Discussion and Conclusion

Compared with alternative methods for CMF skeleton reconstruction, the framework described herein can deal with more types of CMF defects. In the framework described herein, a reliable initial normal model (e.g., the reference bone model) can be estimated from pre-traumatic face photographs, and the initial estimation can be refined by applying both SSM and geometric deformation (AFDSM) techniques. In this way, the advantages of both SSM and geometric deformation are combined, thus eliminating the limitations of using solely one of them, which eventually improved the generalization capacity of the method described herein to address various kinds of CMF defects. Similar to the initial estimation stage of the framework, sparse representation was applied in L. Wang, et al., “Estimating patient-specific and anatomically correct reference model for craniomaxillofacial deformity via sparse representation,” Med. Phys., vol. 42, no. 10, pp. 5809-5816, 2015 to establish a correlation model between midface and jaw, which was then directly applied on the testing patient with remained normal midface to estimate a normal jaw. Compared to the technique of Wang et al. above, the estimation from the correlation model was further refined with the patient's current facial bone, in this way, the estimation accuracy can be improved, which was proved by the results from the ablation studies on the synthetic data.

There is no definitive quantitative measures on the success of post-traumatic reconstruction due to its complexity in clinical practices. The current clinical standard for post-traumatic reconstruction is “surgeons do the best and patient accepts surgical outcomes”. Since there is no groundtruth for the normal bone estimated for a post-traumatic patient, thus no existing evaluation metrics can be applied to evaluate the testing performance of our method. In this study, a metric (i.e. recover degree RD) was used to quantitatively evaluate the clinical testing performance for the method. This metric measures the estimation performance on two aspects that how defective parts are restored and how normal parts are remained in the estimated bone. Considering the aim is to restore defective parts and remain normal parts as much as possible, the metric meets such requirements properly. According to the evaluation results on real patient cases, it was observed that the evaluated metric values are consistent with inspection results from the CMF surgeon, that is the larger RD corresponds to the better estimation, this can confirm that the metric defined above is meaningful.

The study above focused on patients with acquired CMF defects. This disclosure contemplates that the method described herein can also be adapted to correct congenital CMF deformities. For example, for the patients with congenital deformities, e.g., cleft lip and palate, their “pre-deformity” portrait photographs are never existed, thus it is not possible to perform initial bone surface estimation based on the 3D face surface reconstructed from the “pre-deformity” face photographs. In this situation, an initial reference model can be estimated based on the landmarks on bony structure, inspired by the method described in L. Wang, et al., “Estimating patient-specific and anatomically correct reference model for craniomaxillofacial deformity via sparse representation,” Med. Phys., vol. 42, no. 10, pp. 5809-5816, 2015, where the landmarks located on the remaining normal part of the patient's facial bone are used to predict the normal landmark locations on the deformed part of the face. Following the initial estimation, the refinement stage with AFDSM application can be conducted to produce an optimal reference shape model.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method, comprising:
receiving a two-dimensional (“2D”) pre-trauma image of a subject;
generating a three-dimensional (“3D”) facial surface model for the subject from the 2D pre-trauma image;
providing a correlation model between 3D facial and bone surfaces; and
estimating a reference bone model for the subject using the 3D facial surface model and the correlation model.

2. The method of claim 1, wherein the 3D facial surface model is a 3D facial model of the subject's pre-trauma facial soft tissue.

3. The method of any one of claim 1 or 2, wherein the reference bone model is a 3D model of the subject's pre-trauma facial bone structure.

4. The method of any one of claims 1-3, further comprising receiving a plurality of 2D pre-trauma images of the subject, wherein the 3D facial surface model is generated from the plurality of 2D pre-trauma images.

5. The method of claim 4, wherein generating a three-dimensional (“3D”) facial surface model for the subject from the 2D pre-trauma image comprises generating a respective 3D facial surface model for the subject from each of the 2D pre-trauma images and merging the respective 3D facial surface models for the subject into a combined 3D facial surface model.

6. The method of any one of claims 1-5, wherein generating a three-dimensional (“3D”) facial surface model for the subject from the 2D pre-trauma image comprises analyzing the 2D pre-trauma image with a machine learning algorithm.

7. The method of claim 6, wherein generating a three-dimensional (“3D”) facial surface model for the subject from the 2D pre-trauma image comprises:
extracting, using a first machine learning algorithm, a plurality of landmarks from the 2D pre-trauma image; and
generating, using a second machine learning algorithm, the 3D facial surface model from the extracted landmarks.

8. The method of any one of claims 1-5, wherein generating a three-dimensional (“3D”) facial surface model for the subject from the 2D pre-trauma image comprises:
extracting, using a convolutional neural network (“CNN”), a plurality of landmarks from the 2D pre-trauma image;
computing a sparse deformation field between the extracted landmarks and corresponding extracted landmarks for a reference 3D face model;
converting the sparse deformation field into a dense deformation field; and
applying the dense deformation field on the reference 3D face model to generate the 3D facial surface model.

9. The method of any one of claims 1-8, wherein estimating a reference bone model for the subject using the 3D facial surface model and the correlation model comprises:
registering the 3D facial surface model and a facial image template;
mapping a plurality of sample points on the facial image template to the 3D facial surface model to obtain a plurality of corresponding sample points on the 3D facial surface model; and
inputting a coordinate vector of the corresponding sample points into the correlation model to estimate the reference bone model for the subject.

10. The method of claim 9, wherein the facial image template is selected from a plurality of facial surface models.

11. The method of any one of claim 9 or 10, wherein the 3D facial surface model and the facial image template are registered using an iterative closest point (“ICP”) algorithm.

12. The method of any one of claims 9-11, wherein the corresponding sample points on the 3D facial surface model are obtained using a coherent point drift (“CPD”) algorithm.

13. The method of any one of claims 1-12, further comprising:
maintaining a database comprising a plurality of facial and bone surface models, wherein each of the facial and bone surface models is extracted from a 3D image of a reference subject; and
establishing the correlation model based on the facial and bone surface models in the database.

14. The method of claim 13, wherein the correlation model is established using a machine learning algorithm.

15. The method of claim 14, wherein the machine learning algorithm is sparse representation.

16. The method of any one of claims 13-15, wherein the correlation model comprises a face dictionary, the face dictionary comprising a matrix including a plurality of vectorized coordinates of a plurality of landmarks from each of the facial surface models in the database.

17. The method of any one of claims 13-16, wherein the correlation model comprises a bone dictionary, the bone dictionary comprising a matrix representing a plurality of vectorized coordinates of a plurality of landmarks on each of the bone surface models in the database.

18. The method of any one of claims 1-17, further comprising:
receiving a three-dimensional (“3D”) post-trauma image of the subject;
generating a 3D post-trauma bone model from the 3D post-trauma image; and
refining the reference bone model by deforming the reference bone model onto the 3D post-trauma bone model.

19. The method of claim 18, further comprising constraining the reference bone model refinement using a statistical shape model (“SSM”).

20. The method of any one of claim 18 or 19, wherein the reference bone model is refined using a machine learning algorithm.

21. The method of claim 20, wherein the machine learning algorithm is an adaptive-focus deformable shape model (“AFDSM”).

22. The method of any one of claims 18-21, wherein the 3D post-trauma image is a computed-tomography (“CT”) image.

23. The method of any one of claims 1-22, wherein the 2D pre-trauma image is a digital photo.

24. The method of any one of claims 1-23, wherein the subject has craniomaxillofacial (“CMF”) defects.

25. The method of claim 24, wherein the CMF defects are bilateral.

26. The method of any one of claims 1-25, further comprising using the reference bone model for reconstructive surgical planning.

27. A system, comprising:
a processing unit; and
a memory operably coupled to the processing unit, the memory having computer-executable instructions stored thereon that, when executed by the processing unit, cause the processing unit to:
receive a two-dimensional (“2D”) pre-trauma image of a subject;
generate a three-dimensional (“3D”) facial surface model for the subject from the 2D pre-trauma image;
provide a correlation model between 3D facial and bone surfaces; and
estimate a reference bone model for the subject using the 3D facial surface model and the correlation model.

28. The system of claim 27, wherein the 3D facial surface model is a 3D facial model of the subject's pre-trauma facial soft tissue.

29. The system of any one of claim 27 or 28, wherein the reference bone model is a 3D model of the subject's pre-trauma facial bone structure.

30. The system of any one of claims 27-29, wherein the memory has further computer-executable instructions stored thereon that, when executed by the processing unit, cause the processing unit to receive a plurality of 2D pre-trauma images of the subject, wherein the 3D facial surface model is generated from the plurality of 2D pre-trauma images.

31. The system of claim 30, wherein generating a three-dimensional (“3D”) facial surface model for the subject from the 2D pre-trauma image comprises generating a respective 3D facial surface model for the subject from each of the 2D pre-trauma images and merging the respective 3D facial surface models for the subject into a combined 3D facial surface model.

32. The system of any one of claims 27-31, wherein generating a three-dimensional (“3D”) facial surface model for the subject from the 2D pre-trauma image comprises analyzing the 2D pre-trauma image with a machine learning algorithm.

33. The system of claim 32, wherein generating a three-dimensional (“3D”) facial surface model for the subject from the 2D pre-trauma image comprises:
extracting, using a first machine learning algorithm, a plurality of landmarks from the 2D pre-trauma image; and
generating, using a second machine learning algorithm, the 3D facial surface model from the extracted landmarks.

34. The system of any one of claims 27-31, wherein generating a three-dimensional (“3D”) facial surface model for the subject from the 2D pre-trauma image comprises:
extracting, using a convolutional neural network (“CNN”), a plurality of landmarks from the 2D pre-trauma image;
computing a sparse deformation field between the extracted landmarks and corresponding extracted landmarks for a reference 3D face model;
converting the sparse deformation field into a dense deformation field; and
applying the dense deformation field on the reference 3D face model to generate the 3D facial surface model.

35. The system of any one of claims 27-34, wherein estimating a reference bone model for the subject using the 3D facial surface model and the correlation model comprises:
registering the 3D facial surface model and a facial image template;
mapping a plurality of sample points on the facial image template to the 3D facial surface model to obtain a plurality of corresponding sample points on the 3D facial surface model; and
inputting a coordinate vector of the corresponding sample points into the correlation model to estimate the reference bone model for the subject.

36. The system of claim 35, wherein the facial image template is selected from a plurality of facial surface models.

37. The system of any one of claim 35 or 36, wherein the 3D facial surface model and the facial image template are registered using an iterative closest point (“ICP”) algorithm.

38. The system of any one of claims 35-37, wherein the corresponding sample points on the 3D facial surface model are obtained using a coherent point drift (“CPD”) algorithm.

39. The system of any one of claims 27-38, wherein the memory has further computer-executable instructions stored thereon that, when executed by the processing unit, cause the processing unit to:
maintain a database comprising a plurality of facial and bone surface models, wherein each of the facial and bone surface models is extracted from a 3D image of a reference subject; and
establish the correlation model based on the facial and bone surface models in the database.

40. The system of claim 39, wherein the correlation model is established using a machine learning algorithm.

41. The system of claim 40, wherein the machine learning algorithm is sparse representation.

42. The system of any one of claims 39-41, wherein the correlation model comprises a face dictionary, the face dictionary comprising a matrix including a plurality of vectorized coordinates of a plurality of landmarks from each of the facial surface models in the database.

43. The system of any one of claims 39-42, wherein the correlation model comprises a bone dictionary, the bone dictionary comprising a matrix representing a plurality of vectorized coordinates of a plurality of landmarks on each of the bone surface models in the database.

44. The system of any one of claims 27-43, wherein the memory has further computer-executable instructions stored thereon that, when executed by the processing unit, cause the processing unit to:
receive a three-dimensional (“3D”) post-trauma image of the subject;
generate a 3D post-trauma bone model from the 3D post-trauma image; and
refine the reference bone model by deforming the reference bone model onto the 3D post-trauma bone model.

45. The system of claim 44, wherein the memory has further computer-executable instructions stored thereon that, when executed by the processing unit, cause the processing unit to constrain the reference bone model refinement using a statistical shape model (“SSM”).

46. The system of any one of claim 44 or 45, wherein the reference bone model is refined using a machine learning algorithm.

47. The system of claim 46, wherein the machine learning algorithm is an adaptive-focus deformable shape model (“AFDSM”).

48. The system of any one of claims 44-47, wherein the 3D post-trauma image is a computed-tomography (“CT”) image.

49. The system of any one of claims 27-48, wherein the 2D pre-trauma image is a digital photo.

50. The system of any one of claims 27-49, wherein the subject has craniomaxillofacial (“CMF”) defects.

51. The system of claim 50, wherein the CMF defects are bilateral.

52. The system of any one of claims 27-51, wherein the memory has further computer-executable instructions stored thereon that, when executed by the processing unit, cause the processing unit to use the reference bone model for reconstructive surgical planning.

53. A system, comprising:
a first machine learning module configured to analyze two-dimensional (“2D”) images;
a second machine learning module configured to generate a three-dimensional (“3D”) model; and
a processing unit and a memory operably coupled to the processing unit, the memory having computer-executable instructions stored thereon that, when executed by the processing unit, cause the processing unit to:
receive a 2D pre-trauma image of a subject;
input the 2D pre-trauma image into the first machine learning module, the first machine learning module extracting a plurality of landmarks from the 2D pre-trauma image;
input the extracted landmarks into the second machine learning module, the second machine learning module generating a 3D facial surface model for the subject;
provide a correlation model between 3D facial and bone surfaces; and
estimate a reference bone model for the subject using the 3D facial surface model and the correlation model.

54. The system of claim 53, wherein the first machine learning module is a convolutional neural network (“CNN”).

55. The system of any one of claim 53 or 54, wherein the second machine learning module is a module configured to execute thin plate spline (“TPS”).

56. The system of any one of claims 53-55, further comprising a third machine learning module configured to refine the reference bone model, wherein the memory has further computer-executable instructions stored thereon that, when executed by the processing unit, cause the processing unit to:
receive a three-dimensional (“3D”) post-trauma image of the subject;
generate a 3D post-trauma bone model from the 3D post-trauma image; and
input the reference bone model into the third machine learning module to deform the reference bone model onto the 3D post-trauma bone model.

57. The system of claim 56, wherein the memory has further computer-executable instructions stored thereon that, when executed by the processing unit, cause the processing unit to constrain the reference bone model refinement using a statistical shape model (“SSM”).

58. The system of any one of claim 56 or 57, wherein the third machine learning module is a module configured to execute an adaptive-focus deformable shape model (“AFDSM”).
